(19) United States
(12) Patent Application Publication
(10) Pub. No.: US 2015/0118244 A1
(43) Pub. Date: Apr. 30, 2015

(54) ANTI-TUMOR ANTIBODIES AS PREDICTIVE OR PROGNOSTIC BIOMARKERS OF EFFICACY AND SURVIVAL IN IPILIMUMAB-TREATED PATIENTS

(71) Applicant: BRISTOL-MYERS SQUIBB COMPANY, Princeton, NJ (US)

(72) Inventors: Vafa Shahabi, Valley Forge, PA (US); Rui-Ru Ji, Princeton, NJ (US)

(73) Assignee: Bristol-Myers Squibb Company, Princeton, NJ (US)

(21) Appl. No.: 14/399,554

(22) PCT Filed: May 9, 2013
(86) PCT No.: PCT/US2013/040256

§ 371 Date: Nov. 7, 2014

Related U.S. Application Data
(60) Provisional application No. 61/645,109, filed on May 10, 2012.
(60) Provisional application No. 61/653,802, filed on May 31, 2012.

(51) Int. Cl. G01N 33/574; C07K 16/28

(52) U.S. Cl. CPC G01N 33/57484; C07K 16/2818
USPC 424/142.1; 506/9

(57) ABSTRACT

Provided herein are prognostic and diagnostic methods and kits for use with the methods. For example, provided herein are methods for determining whether a subject having cancer will respond to a cancer treatment. For example, provided herein are methods for determining whether a subject having advanced melanoma will respond to a treatment with ipilimumab. Methods for determining the length of survival of cancer patients, e.g., melanoma patients, such as melanoma patients treated with ipilimumab, are also provided herein.

### CROSS-REFERENCE TO RELATED APPLICATIONS

This application claims priority to U.S. provisional application No. 61/645,109, filed on May 10, 2012 and U.S. provisional application No. 61/653,802, filed on May 31, 2012, both of which are specifically incorporated by reference herein in their entirety.

### SEQUENCE LISTING

The instant application contains a Sequence Listing, which is been submitted via EFS-Web concurrently with the instant application, and is hereby incorporated by reference in its entirety. Said Sequence Listing is in text format, was created on May 7, 2013, is named “11966-WO-PCT Sequence Listing_ST25.txt” and is 38,076 bytes in size.

### BACKGROUND

The National Cancer Institute has estimated that in the United States alone, 1 in 3 people will be struck with cancer during their lifetime. Moreover, approximately 50% to 60% of people contracting cancer will eventually succumb to the disease. The widespread occurrence of this disease underscores the need for improved anticancer regimens for the treatment of malignancy.

Due to the wide variety of cancers presently observed, numerous anticancer agents have been developed to destroy cancer within the body. These compounds are administered to cancer patients with the objective of destroying or otherwise inhibiting the growth of malignant cells while leaving normal, healthy cells undisturbed. Anticancer agents have been classified based upon their mechanism of action, and are often referred to as chemotherapeutics, or immunotherapeutics (agents whose therapeutic effects are mediated by their immuno-modulating properties). The vertebrate immune system requires multiple signals to achieve optimal immune activation; see, e.g., Janeway, Cold Spring Harbor Symp. Quant. Biol., 54:1-14 (1989); Paul, W. E., ed., Fundamental Immunology, 4th Edition, Raven Press, NY (1998), particularly Chapters 12 and 13, pp. 411-478. Interactions between T lymphocytes (T cells) and antigen presenting cells (APC's) are essential to the immune response. Levels of many cohesive molecules found on T cells and APC's increase during an immune response (Springer et al., Ann. Rev. Immunol., 5:223-252 (1987); Shaw et al., Curr. Opin. Immunol., 1:92-97 (1988)); and Hemler, Immunology Today, 9:109-113 (1988)). Increased levels of these molecules may help explain why activated APC's are more effective at stimulating antigen-specific T cell proliferation than are resting APC's (Kaiuchi et al., J. Immunol., 131:109-114 (1983); Kreiger et al., J. Immunol., 135:2937-2945 (1985); McKenzie, J. Immunol., 141:2907-2911 (1988); and Hawrylowicz et al., J. Immunol., 141:4083-4088 (1988)).

T cell immune response is a complex process that involves cell-cell interactions (Springer et al., Ann. Rev. Immunol., 5:223-252 (1987)), particularly between T and accessory cells such as APC's, and production of soluble immune mediators (cytokines or lymphokines) (Dinarello, New Engl. J. Med., 317:940-945 (1987); Sallusto, J. Exp. Med., 179:1109-1118 (1997)). This response is regulated by several T-cell surface receptors, including the T-cell receptor complex (Weiss, Ann. Rev. Immunol., 4:593-619 (1986)) and other “accessory” surface molecules (Allison, Curr. Opin. Immunol., 6:414-419 (1994); Springer (1987), supra). Many of these accessory molecules are naturally occurring cell surface differentiation (CD) antigens defined by the reactivity of monoclonal antibodies on the surface of cells (McMichael, ed., Leukocyte Typing Iff, Oxford Univ. Press, Oxford, N.Y. (1987)).

Early studies suggested that B lymphocyte activation requires two signals (Bretscher, Science, 169:1042-1049 (1970)) and now it is believed that all lymphocytes require two signals for their optimal activation, an antigen specific or clonal signal, as well as a second, antigen non-specific signal. (Janeway, supra). Freeman (J. Immunol., 143:2714-2722 (1989)) isolated and sequenced a cDNA clone encoding a B cell activation antigen recognized by MAb B7 (Freeman, J. Immunol., 139:3260 (1987)). COS cells transfected with this cDNA have been shown to stain by both labeled MAb B7 and MAb BB-1 (Clark, Human Immunol., 16:100-113 (1986); Yokochi, J. Immunol., 128:823 (1981); Freeman et al. (1989), supra; Freeman et al. (1987), supra). In addition, expression of this antigen has been detected on cells of other lineages, such as monocytes (Freeman et al. (1989), supra).

T helper cell (Th) antigenic response requires signals provided by APC's. The first signal is initiated by interaction of the T cell receptor complex (Weiss, J. Clin. Invest., 86:1015 (1990)) with antigen presented in the context of major histocompatibility complex (MHC) molecules on the APC (Allen, Immunol. Today, 8:270 (1987)). This antigen-specific signal is not sufficient to generate a full response, and in the absence of a second signal may actually lead to clonal inactivation or anergy (Schwartz, Science, 248:1349 (1990)). The requirement for a second “costimulatory” signal has been demonstrated in a number of experimental systems (Schwartz, supra; Weaver et al., Immunol. Today, 11:49 (1990)).

CD28 antigen, a homodimeric glycoprotein of the immunoglobulin superfamily (Aruffo et al., Proc. Natl. Acad. Sci., 84:8573-8577 (1987)), is an accessory molecule found on most mature human T cells (Damle et al., J. Immunol., 131:2296-2300 (1983)). Current evidence suggests that this molecule functions in an alternative T cell activation pathway distinct from that initiated by the T-cell receptor complex (June et al., Mol. Cell. Biol., 7:4472-4481 (1987)). Monoclonal antibodies (MAbs) reactive with CD28 antigen can augment T cell responses initiated by various polyclonal stimuli (reviewed by June et al., supra). These stimulatory effects may result from MAb-induced cytokine production (Thompson et al., Proc. Natl. Acad. Sci., 86:1333-1337 (1989); and Lindsten et al., Science, 244:339-343 (1989)) as a consequence of increased mRNA stabilization (Lindsten et al. (1989), supra). Anti-CD28 mAbs can also have inhibitory effects, i.e., they can block autologous mixed lymphocyte reactions (Damle et al., Proc. Natl. Acad. Sci., 78:5096-6001 (1981)) and activation of antigen-specific T cell clones (Lesslauer et al., Eur. J. Immunol., 16:1289-1296 (1986)).

Some studies have indicated that CD28 is a counter-receptor for the B cell activation antigen, B7/BB-1 (Linsley et al., Proc. Natl. Acad. Sci. USA, 87:5031-5035 (1990)). The B7/BB-1 antigen is hereafter referred to as the “B7 antigen”. The B7 ligands are also members of the immunoglobulin superfamily but have, in contrast to CD28, two Ig domains in their extracellular region, an N-terminal variable (V)-like domain followed by a constant (C)-like domain.

Delivery of a non-specific costimulatory signal to the T cell requires at least two homologous B7 family members found on APC's, B7-1 (also called B7, B7.1, or CD80) and B7-2 (also called B7.2 or CD86), both of which can deliver costimulatory signals to T cells via CD28. Costimulation through CD28 promotes T cell activation.

CD28 has a single extracellular variable region (V)-like domain (Aruffo et al., supra). A homologous molecule, CTLA-4, has been identified by differential screening of a murine cytolytic-T cell cDNA library (Brunet, Nature, 328:267-270 (1987)).

CTLA-4 (CD152) is a T cell surface molecule that was originally identified by differential screening of a murine cytolytic T cell cDNA library (Brunet et al., Nature, 328:267-270 (1987)). CTLA-4 is also a member of the immunoglobulin (Ig) superfamily; CTLA-4 comprises a single extracellular Ig domain. Researchers have reported the cloning and mapping of a gene for the human counterpart of CTLA-4 (Dariavach et al., Eur. J. Immunol., 18:1901-1905 (1988)) to the same chromosomal region (2q33-34) as CD28 (Lafage-Pochitaloff et al., Immunogenetics, 31:198-201 (1990)). Sequence comparison between this human CTLA-4 DNA and that encoding CD28 proteins reveals significant homology of sequence, with the greatest degree of homology in the juxtamembrane and cytoplasmic regions (Brunet et al. (1988), supra; Dariavach et al. (1988), supra).

The CTLA-4 is inducibly expressed by T cells. It binds to the B7-family of molecules (primarily CD80 and CD86) on antigen-presenting cells (Chambers et al., Ann. Rev. Immunol., 19:565-594 (2001)). When triggered, it inhibits T-cell proliferation and function. Mice genetically deficient in CTLA-4 develop lymphoproliferative disease and autoimmunity (Tivol et al., Immunity, 3:541-547 (1995)). In pre-clinical models, CTLA-4 blockade also augments anti-tumor immunity (Leach et al., Science, 271:1734-1736 (1996); van Elsas et al., J. Exp. Med., 190:355-366 (1999)). These findings led to the development of antibodies that block CTLA-4 for use in cancer immunotherapy.

Blockade of CTLA-4 by a monoclonal antibody leads to the expansion of all T cell populations, with activated CD4⁺ and CD8⁺ T cells mediating tumor cell destruction (Melero et al., Nat. Rev. Cancer, 7:95-106 (2007); Wolchok et al., The Oncologist, 13(Suppl. 4):2-9 (2008)). The antitumor response that results from the administration of anti-CTLA-4 antibodies is believed to be due to an increase in the ratio of effector T cells to regulatory T cells within the tumor microenvironment, rather than simply from changes in T cell populations in the peripheral blood (Quezada et al., J. Clin. Invest., 116:1935-1945 (2006)). One such agent is ipilimumab.

Ipilimumab (previously MDX-010; Medarex Inc., marketed by Bristol-Myers Squibb as YERVOY™) is a fully human anti-human CTLA-4 monoclonal antibody that blocks the binding of CTLA-4 to CD80 and CD86 expressed on antigen presenting cells, thereby, blocking the negative down-regulation of the immune responses elicited by the interaction of these molecules. Initial studies in patients with melanoma showed that ipilimumab could cause objective durable tumor regressions (Phan et al., Proc. Natl. Acad. Sci. USA, 100:8372-8377 (2003)). Also, reductions of serum tumor markers such as CA125 and PSA were seen for some patients with ovarian or prostate cancer, respectively (Hodi et al., Proc. Natl. Acad. Sci. USA, 100:4712-4717 (2003)). Ipilimumab has demonstrated antitumor activity in patients with advanced melanoma (Weber et al., J. Clin. Oncol., 26:5950-5956 (2008); Weber, Cancer Immunol. Immunother., 58:823-830 (2009)). In addition in a number of phase II and two phase III clinical trials ipilimumab was shown to increase the overall survival in advanced melanoma patients (Hodi, F. S. et al., “Improved survival with ipilimumab in patients with metastatic melanoma”, New Engl. J. Med., 363:711-723 (2010), and Robert, C. et al., “Ipilimumab plus dacarbazine for previously untreated metastatic melanoma”, New Engl. J. Med., 364:2517-2526 (2011)).

### SUMMARY

Provided herein are methods for treating a subject having cancer. A method may comprise identifying a subject having cancer and having a level of antibodies to each of at least two tumor associated antigens (TAAs) that is higher than a predetermined antibody value for each of the two TAAs; and administering to the subject a therapeutically effective amount of a therapeutic agent for treating cancer. In one embodiment, a method is for treating a subject having melanoma and the method comprises identifying a subject having melanoma and having a level of antibodies to each of at least two tumor associated antigens (TAAs) that is higher than a predetermined antibody value for each of the two TAAs; and administering to the subject a therapeutically effective amount of a therapeutic agent for treating melanoma. In certain embodiments, the at least two TAAs are selected from the group consisting of CTAG2, SSX2, SPANXA1, NLRP4, PBK and SPANXB1. The at least two TAAs may be selected from the group consisting of CTAG2, SSX2, SPANXA1, NLRP4 and PBK. The at least two TAAs may be selected from the group consisting of CTAG2, SSX2 and SPANXA1. The therapeutic agent for treating melanoma may be an immunotherapeutic agent, such as an anti-CTLA4 agent (e.g., antibody or antigen binding portion thereof), e.g., ipilimumab. In certain embodiments, the subject has advanced melanoma, such as metastatic melanoma, e.g., stage III or IV melanoma. The melanoma may be unresectable stage III or IV melanoma. A method may comprise obtaining a serum sample from the subject and determining the level of antibodies to the at least two TAAs in the serum sample. The subject may be a subject who is not being treated with ipilimumab at the time the level of antibodies of each of the at least two TAAs is determined in the subject. An exemplary method for treating a subject having advanced melanoma with ipilimumab comprises identifying a subject having advanced melanoma and having a level of antibodies to each of at least two TAAs selected from the group consisting of CTAG2, SSX2, SPANXA1, NLRP4, PBK and SPANXB1 that is higher than a predetermined antibody value for each of the two TAAs; and administering to the subject a therapeutically effective amount of ipilimumab.

Also provided herein are methods for treating a subject having cancer with ipilimumab, comprising identifying a subject having cancer and having a level of antibodies to each of at least two TAAs selected from the group consisting of CTAG2, SSX2, SPANXA1, NLRP4, PBK and SPANXB1 that is higher than a predetermined antibody value for each of the two TAAs; and administering to the subject a therapeutically effective amount of ipilimumab. The at least two TAAs may be selected from the group consisting of CTAG2, SSX2, SPANXA1, NLRP4 and PBK. The at least two TAAs may be selected from the group consisting of CTAG2, SSX2 and SPANXA1. The subject may be a subject having advanced cancer. The subject may be a subject having prostate cancer, pancreatic cancer, lung cancer, breast cancer, colon cancer, urothelial carcinoma, lymphoma or leukemia. A method may comprise obtaining a serum sample from the subject and determining the level of antibodies to the at least two TAAs in the serum sample. In certain embodiments, the subject is not being treated with ipilimumab at the time the level of antibodies to each of at least two TAAs is determined in the subject.

Also provided herein are methods for treating a subject having cancer, comprising determining the level of antibodies to each of at least two TAAs in a subject having cancer; and administering to the subject a therapeutically effective dose of a therapeutic agent for treating cancer if the level of antibodies to each of at least two TAAs in the subject is higher than a predetermined antibody value for each of the two TAAs. In one embodiment, a method is for treating a subject having melanoma and the method comprises determining the level of antibodies to each of at least two TAAs in a subject having melanoma; and administering to the subject a therapeutically effective dose of a therapeutic agent for treating melanoma if the level of antibodies to each of at least two TAAs in the subject is higher than a predetermined antibody value for each of the two TAAs. The at least two TAAs may be selected from the group consisting of CTAG2, SSX2, SPANXA1, NLRP4, PBK and SPANXB1. The at least two TAAs may be selected from the group consisting of CTAG2, SSX2, SPANXA1, NLRP4 and PBK. The at least two TAAs are selected from the group consisting of CTAG2, SSX2 and SPANXA1. The therapeutic agent for treating melanoma may be an immunotherapeutic agent, such as an anti-CTLA4 agent (e.g., antibody or an antigen binding portion thereof), e.g., ipilimumab. The subject may be a subject having advanced melanoma, such as metastatic melanoma, e.g., stage III or IV melanoma. The subject may also have unresectable stage III or IV melanoma. The method may comprise obtaining a serum sample from the subject and determining the level of antibodies to the at least two TAAs in the serum sample. In certain embodiments, the subject is not being treated with ipilimumab at the time the level of antibodies to each of the at least two TAAs is determined in the subject. In an exemplary embodiment, a method for treating a subject having advanced melanoma with ipilimumab comprises determining the level of antibodies to each of at least two TAAs selected from the group consisting of CTAG2, SSX2, SPANXA1, NLRP4, PBK and SPANXB1 in a subject having melanoma; and administering to the subject a therapeutically effective dose of ipilimumab if the level of antibodies to each of at least two TAAs in the subject is higher than a predetermined antibody value for each of the two TAAs.

Also encompassed herein are methods for treating a subject having cancer with ipilimumab, comprising determining the level of antibodies to each of at least two TAAs selected from the group consisting of CTAG2, SSX2, SPANXA1, NLRP4, PBK and SPANXB1 in a subject having cancer; and administering to the subject a therapeutically effective dose of ipilimumab if the level of antibodies to each of at least two TAAs in the subject is higher than a predetermined antibody value for each of the two TAAs. The at least two TAAs may be selected from the group consisting of CTAG2, SSX2, SPANXA1, NLRP4 and PBK. The at least two TAAs may be selected from the group consisting of CTAG2, SSX2 and SPANXA1. The subject may be a subject having advanced cancer. The subject may be a subject having prostate cancer, pancreatic cancer, lung cancer, breast cancer, colon cancer, urothelial carcinoma, lymphoma or leukemia. A method may comprise obtaining a serum sample from the subject and determining the level of antibodies to each of the at least two TAAs in the serum sample. In certain embodiments, the subject is not being treated with ipilimumab at the time the level of antibodies of each of the at least two TAAs is determined in the subject.

Further provided herein are methods for determining whether a subject having cancer is likely to respond to a therapeutic agent for treating cancer. A method may comprise determining the level of antibodies to each of at least two TAAs in a subject having cancer, wherein (i) a higher level of antibodies to each of at least two TAAs in the subject having cancer relative to a predetermined antibody value for each TAA indicates that the subject is likely to respond to a therapeutic agent for treating cancer; and (ii) the absence of a higher level of antibodies to each of at least two TAAs in the subject having cancer relative to a predetermined antibody value for each TAA indicates that the subject is not likely to respond to a therapeutic agent for treating cancer. In one embodiment, a method is for determining whether a subject having melanoma is likely to respond to a therapeutic agent for treating melanoma, and the method comprises determining the level of antibodies to each of at least two TAAs in a subject having melanoma, wherein (i) a higher level of antibodies to each of at least two TAAs in the subject having melanoma relative to a predetermined antibody value for each TAA indicates that the subject is likely to respond to a therapeutic agent for treating melanoma; and (ii) the absence of a higher level of antibodies to each of at least two TAAs in the subject having melanoma relative to a predetermined antibody value for each TAA indicates that the subject is not likely to respond to a therapeutic agent for treating melanoma. The at least two TAAs may be selected from the group consisting of CTAG2, SSX2, SPANXA1, NLRP4, PBK and SPANXB1. The at least two TAAs may be selected from the group consisting of CTAG2, SSX2, SPANXA1, NLRP4 and PBK. The at least two TAAs may be selected from the group consisting of CTAG2, SSX2 and SPANXA1. The therapeutic agent for treating melanoma may be an immunotherapeutic agent, such as an anti-CTLA4 agent (e.g., antibody or an antigen binding portion thereof), e.g., ipilimumab. The subject may have advanced melanoma, such as metastatic melanoma, e.g., stage III or IV melanoma. The subject may have unresectable stage III or IV melanoma. The method may comprise obtaining a serum sample from the subject and determining the level of antibodies to the at least two TAAs in the serum sample. In certain embodiments, the subject is not being treated with ipilimumab at the time the level of antibodies of each of the at least two TAAs is determined in the subject. In an exemplary embodiment, a method for determining whether a subject having advanced melanoma is likely to respond to treatment with ipilimumab comprises determining the level of antibodies to the at least two TAAs selected from the group consisting of CTAG2, SSX2, SPANXA1, NLRP4, PBK and SPANXB1 in a subject having advanced melanoma, wherein (i) a higher level of antibodies to each of at least two TAAs in the subject having advanced melanoma relative to a predetermined antibody value for each TAA indicates that the subject is likely to respond to treatment with ipilimumab; and (ii) the absence of a higher level of antibodies to each of at least two TAAs in the subject having advanced melanoma relative to a predetermined antibody value for each TAA indicates that the subject is not likely to respond to treatment with ipilimumab.

Also provided herein are methods for determining whether a subject having cancer is likely to respond to treatment with ipilimumab. A method may comprise determining the level of antibodies to each of at least two TAAs in a subject having cancer, wherein (i) a higher level of antibodies to each of at least two TAAs selected from the group consisting of CTAG2, SSX2, SPANXA1, NLRP4, PBK and SPANXB1 in the subject having cancer relative to a predetermined antibody value for each TAA indicates that the subject is likely to respond to treatment with ipilimumab; and (ii) the absence of a higher level of antibodies to each of at least two TAAs selected from the group consisting of CTAG2, SSX2, SPANXA1, NLRP4, PBK and SPANXB1 in the subject having cancer relative to a predetermined antibody value for each TAA indicates that the subject is not likely to respond to treatment with ipilimumab. The at least two TAAs may be selected from the group consisting of CTAG2, SSX2, SPANXA1, NLRP4 and PBK. The at least two TAAs may be selected from the group consisting of CTAG2, SSX2 and SPANXA1. The subject may be a subject having advanced cancer. The subject may be a subject having prostate cancer, pancreatic cancer, lung cancer, breast cancer, colon cancer, urothelial carcinoma, lymphoma or leukemia. The method may comprise obtaining a serum sample from the subject and determining the level of antibodies to the at least two TAAs in the serum sample. In certain embodiments, the subject is not being treated with ipilimumab at the time the level of antibodies of each of the at least two TAAs is determined in the subject.

Also encompassed herein are methods for determining whether to treat a subject having cancer with a therapeutic agent for cancer. A method may comprise determining the level of antibodies to each of at least two TAAs in a subject having cancer, wherein (i) a higher level of antibodies to each of at least two TAAs relative to a predetermined antibody value for each of the TAAs indicates that the subject should be treated with a therapeutic agent for cancer; and (ii) the absence of a higher level of antibodies to each of at least two TAAs relative to a predetermined antibody value for each of the TAAs indicates that the subject should not be treated with a therapeutic agent for cancer. In one embodiment, the method is for determining whether to treat a subject having melanoma with a therapeutic agent for melanoma, and the method comprises determining the level of antibodies to each of at least two TAAs in a subject having melanoma, wherein (i) a higher level of antibodies to each of at least two TAAs relative to a predetermined antibody value for each of the TAAs indicates that the subject should be treated with a therapeutic agent for melanoma; and (ii) the absence of a higher level of antibodies to each of at least two TAAs relative to a predetermined antibody value for each of the TAAs indicates that the subject should not be treated with a therapeutic agent for melanoma. The at least two TAAs may be selected from the group consisting of CTAG2, SSX2, SPANXA1, NLRP4, PBK and SPANXB1. The at least two TAAs may be selected from the group consisting of CTAG2, SSX2, SPANXA1, NLRP4 and PBK. The at least two TAAs may be selected from the group consisting of CTAG2, SSX2 and SPANXA1. The therapeutic agent for treating melanoma may be an immunotherapeutic agent, such as an anti-CTLA4 agent (e.g., an antibody or an antigen binding portion thereof), e.g., ipilimumab. The subject may be a subject having advanced melanoma, such as metastatic melanoma, e.g., stage III or IV melanoma. A subject may have unresectable stage III or IV melanoma. A method may comprise obtaining a serum sample from the subject and determining the level of antibodies to the at least two TAAs in the serum sample. In certain embodiments, the subject is not being treated with ipilimumab at the time the level of antibodies of each of the at least two TAAs is determined in the subject. In an exemplary embodiment, a method for determining whether to treat a subject having advanced melanoma with ipilimumab comprises determining the level of antibodies to each of at least two TAAs selected from the group consisting of CTAG2, SSX2, SPANXA1, NLRP4, PBK and SPANXB1 in a subject having advanced melanoma, wherein (i) a higher level of antibodies to each of at least two TAAs relative to a predetermined antibody value for each of the TAAs indicates that the subject should be treated with ipilimumab; and (ii) the absence of a higher level of antibodies to each of at least two TAAs relative to a predetermined antibody value for each of the TAAs indicates that the subject should not be treated with ipilimumab.

Further encompassed herein are methods for determining whether to treat a subject having cancer with ipilimumab, comprising determining the level of antibodies to each of at least two TAAs selected from the group consisting of CTAG2, SSX2, SPANXA1, NLRP4, PBK and SPANXB1 in a subject having cancer, wherein (i) a higher level of antibodies to each of two TAAs relative to a predetermined antibody value for each of the TAAs indicates that the subject should be treated with ipilimumab; and (ii) the absence of a higher level of antibodies to each of two TAAs relative to a predetermined antibody value for each of the TAAs indicates that the subject should not be treated with ipilimumab. The at least two TAAs may be selected from the group consisting of CTAG2, SSX2, SPANXA1, NLRP4 and PBK. The at least two TAAs may be selected from the group consisting of CTAG2, SSX2 and SPANXA1. The subject may be a subject having advanced cancer. The subject may be a subject having prostate cancer, pancreatic cancer, lung cancer, breast cancer, colon cancer, urothelial carcinoma, lymphoma or leukemia. The method may comprise obtaining a serum sample from the subject and determining the level of antibodies to the at least two TAAs in the serum sample. In certain embodiments, the subject is not being treated with ipilimumab at the time the level of antibodies of each of the at least two TAAs is determined in the subject.

Also encompassed herein are methods for predicting the length of survival of a subject having cancer, comprising determining the level of antibodies to each of at least two TAAs in a subject having cancer, wherein (i) a higher level of antibodies to each of at least two TAAs in the subject relative to a predetermined antibody value for each TAA indicates that the subject is likely to have a longer survival relative to a subject who does not have a level of antibodies to each of at least two TAAs that is higher than the predetermined antibody value for each TAA; and (ii) the absence of a higher level of antibodies to each of at least two TAAs in the subject relative to a predetermined antibody value for each TAA indicates that the subject is not likely to have a longer survival relative to a subject who has a level of antibodies to each of at least two TAAs that is higher than the predetermined antibody value for each TAA. In one embodiment, the method is a method for predicting the length of survival of a subject having melanoma, and the method comprises determining the level of antibodies to each of at least two TAAs in a subject having melanoma, wherein (i) a higher level of antibodies to each of at least two TAAs in the subject relative to a predetermined antibody value for each TAA indicates that the subject is likely to have a longer survival relative to a subject who does not have a level of antibodies to each of at least two TAAs that is higher than the predetermined antibody value for each TAA; and (ii) the absence of a higher level of antibodies to each of at least two TAAs in the subject relative to a predetermined antibody value for each TAA indicates that the subject is not likely to have a longer survival relative to a subject who has a level of antibodies to each of at least two TAAs that is higher than the predetermined antibody value for each TAA. The at least two TAAs may be selected from the group consisting of CTAG2, SSX2, SPANXA1, NLRP4, PBK and SPANXB1. The at least two TAAs may be selected from the group consisting of CTAG2, SSX2, SPANXA1, NLRP4 and PBK. The at least two TAAs may be selected from the group consisting of CTAG2, SSX2 and SPANXA1. The subject may be a subject having advanced melanoma such as metastatic melanoma, e.g., stage III or IV melanoma. A subject may have unresectable stage III or IV melanoma. A subject having a level of antibodies to each of at least two TAAs that is higher than a predetermined antibody value of each of at least two TAAs is likely to survive at least 300 days, 400 days, 500 days (or 1 year, 2 years) or more longer than a subject having melanoma and who does not have a level of antibodies to each of two TAAs that is higher than a predetermined antibody value of each TAA. The method may comprise obtaining a serum sample from the subject and determining the level of antibodies to the at least two TAAs in the serum sample. In certain embodiments, the subject is not being treated with ipilimumab at the time the level of antibodies of each of the at least two TAAs is determined in the subject. In an exemplary embodiment, a method for predicting the length of survival of a subject having advanced melanoma, comprises determining the level of antibodies to each of at least two TAAs selected from the group consisting of CTAG2, SSX2, SPANXA1, NLRP4, PBK and SPANXB1 in a subject having advanced melanoma, wherein (i) a higher level of antibodies to each of at least two TAAs in the subject relative to a predetermined antibody value for each TAA indicates that the subject is likely to have a longer survival relative to a subject who does not have a level of antibodies to each of at least two TAAs that is higher than the predetermined antibody value for each TAA; and (ii) the absence of a higher level of antibodies to each of at least two TAAs in the subject relative to a predetermined antibody value for each TAA indicates that the subject is not likely to have a longer survival relative to a subject who has a level of antibodies to each of at least two TAAs that is higher than the predetermined antibody value for each TAA. A subject may be treated with a therapeutic agent for melanoma after determining the level of antibodies to each of at least two TAAs. The therapeutic agent for melanoma may be an immunotherapeutic agent, such as an anti-CTLA4 agent (e.g., an antibody or an antigen binding portion thereof), e.g., ipilimumab. An exemplary method for predicting the length of survival of a subject having advanced melanoma and being treated with ipilimumab may comprise determining the level of antibodies to each of at least two TAAs selected from the group consisting of CTAG2, SSX2, SPANXA1, NLRP4, PBK and SPANXB1 in a subject having advanced melanoma prior to the start of the treatment with ipilimumab, wherein (i) a higher level of antibodies to each of at least two TAAs in the subject relative to a predetermined antibody value for each TAA indicates that the subject is likely to have a longer survival relative to a subject who does not have a level of antibodies to each of at least two TAAs that is higher than the predetermined antibody value for each TAA; and (ii) the absence of a higher level of antibodies to each of at least two TAAs in the subject relative to a predetermined antibody value for each TAA indicates that the subject is not likely to have a longer survival relative to a subject who has a level of antibodies to each of at least two TAAs that is higher than the predetermined antibody value for each TAA.

Further provided herein are methods for predicting the length of survival of a subject having cancer, comprising determining the level of antibodies to each of at least two TAAs selected from the group consisting of CTAG2, SSX2, SPANXA1, NLRP4, PBK and SPANXB1 in a subject having cancer, wherein (i) a higher level of antibodies to each of at least two TAAs in the subject relative to a predetermined antibody value for each TAA indicates that the subject is likely to have a longer survival relative to a subject who does not have a level of antibodies to each of at least two TAAs that is higher than the predetermined antibody value for each TAA; and (ii) the absence of a higher level of antibodies to each of at least two TAAs in the subject relative to a predetermined antibody value for each TAA indicates that the subject is not likely to have a longer survival relative to a subject who has a level of antibodies to each of at least two TAAs that is higher than the predetermined antibody value for each TAA. The at least two TAAs may be selected from the group consisting of CTAG2, SSX2, SPANXA1, NLRP4 and PBK. The at least two TAAs may be selected from the group consisting of CTAG2, SSX2 and SPANXA1. The subject may be a subject having advanced cancer. The method may comprise obtaining a serum sample from the subject and determining the level of antibodies to each of the at least two TAAs in the serum sample. In certain embodiments, the subject is not being treated with ipilimumab at the time the level of antibodies of each of the at least two TAAs is determined in the subject. In certain embodiments, the subject is treated with a therapeutic agent for cancer after determining the level of antibodies to each of at least two TAAs. The therapeutic agent for cancer may be an immunotherapeutic agent, such as an anti-CTLA4 agent (e.g., an antibody or antigen binding portion thereof), e.g., ipilimumab. In an exemplary embodiment, a method for predicting the length of survival of a subject having cancer and being treated with ipilimumab comprises determining the level of antibodies to each of at least two TAAs selected from the group consisting of CTAG2, SSX2, SPANXA1, NLRP4, PBK and SPANXB1 in a subject having cancer prior to the start of the treatment with ipilimumab, wherein (i) a higher level of antibodies to each of at least two TAAs in the subject relative to a predetermined antibody value for each TAA indicates that the subject is likely to have a longer survival relative to a subject who does not have a level of antibodies to each of at least two TAAs that is higher than the predetermined antibody value for each TAA; and (ii) the absence of a higher level of antibodies to each of at least two TAAs in the subject relative to a predetermined antibody value for each TAA indicates that the subject is not likely to have a longer survival relative to a subject who has a level of antibodies to each of at least two TAAs that is higher than the predetermined antibody value for each TAA.

Also provided herein are methods for determining the level of antibodies to each of at least two TAAs selected from the group consisting of CTAG2, SSX2, SPANXA1, NLRP4, PBK and SPANXB1 in a serum sample of a human subject, comprising (i) providing a serum sample from a human subject; and (ii) measuring the level of antibodies to each of at least two TAAs selected from the group consisting of CTAG2, SSX2, SPANXA1, NLRP4, PBK and SPANXB1. The subject may be a subject having cancer, such as melanoma, e.g., advanced melanoma, e.g., metastatic melanoma, e.g., stage III or IV melanoma, e.g., unresectable stage III or IV melanoma. The serum sample may be a serum sample from a human subject that is not treated with ipilimumab. The at least two TAAs may be selected from the group consisting of CTAG2, SSX2, SPANXA1, NLRP4 and PBK. The at least two TAAs may be selected from the group consisting of CTAG2, SSX2, and SPANXA1. The at least two TAAs may be a pair of TAAs selected from the pairs of TAAs consisting of CTAG2 and SSX2; CTAG2 and SPANXA1; and SSX2 and SPANXA1. The method may comprise determining the level of antibodies to each of at least three TAAs selected from the group consisting of CTAG2, SSX2, SPANXA1, NLRP4, PBK and SPANXB1. The at least three TAAs may be CTAG2, SSX2 and SPANXA1.

Further encompassed herein are kits. Kits may comprise one or more reagents for determining the level of antibodies to each of at least two TAAs selected from the group consisting of CTAG2, SSX2, SPANXA1, NLRP4, PBK and SPANXB1 in a serum sample, wherein the kit comprises at least two isolated human proteins or antigenic portions thereof selected from the group of human proteins consisting of CTAG2, SSX2, SPANXA1, NLRP4, PBK and SPANXB1, and wherein the kit does not comprise isolated human proteins or antigenic portions thereof that are not useful for determining whether a subject having cancer is likely to respond to a therapy with ipilimumab. The kit may comprise a human CTAG2 protein (e.g., isoform 1) or antigenic fragment thereof, a human SSX2 protein (e.g., isoform a) or antigenic fragment thereof, a human SPANXA1 protein or antigenic fragment thereof, a human NLRP4 protein or antigenic fragment thereof, a human PBK protein or antigenic fragment thereof and a human SPANXB1 protein or antigenic fragment thereof. In certain embodiments, a kit does not comprise any other protein or antigenic fragment thereof. A kit may further comprise a reagent for detecting human antibodies.

### BRIEF DESCRIPTION OF THE DRAWINGS

FIG. 1A: Ab array of CA184-004 baseline serum samples showing reactivity toward 37 analytes covering 30 TAAs. Average Score shows the average value in all the squares above the score. Empty squares were given a value of 0. Numbers indicate the intensity of RFU signals.

FIG. 1B: Baseline Anti-tumor Abs predictive of survival. Ab array of CA184-004 baseline serum samples showing reactivity toward 37 analytes covering 30 TAAs. Average Score shows the average value in all the squares above the score. Empty squares were given a value of 0. Numbers indicate the intensity of RFU signals. Data were sorted based on the patients who received previous anti-cancer treatment (Y) or not (N) before enrolling in the trial.

FIG. 2: ELISA assay on baseline serum samples from CA184-004 and -007. OD₄₅₀ readings of serum dilution of 1:400 is displayed. Last row is the background+3×standard division (SD). Any OD₄₅₀ reading above this value was considered as positive and are colored as a heat map. Heatmap showing the Ab profile in CA184-004 (A) and in CA184-007 (B) of three potential predictive antibodies (CTAG2, SPANXA1 and SSX2). (C, D) Kaplan-Meier (k-M) plot of survival divided by patients showing Ab response to 0-1 antigen or 2-3 antigens in the heatmaps. Tables display the statistical analysis of these K-M plot.

FIG. 3A: Effect of ipilimumab on Ig gene expression in peripheral blood. mRNA was isolated from peripheral blood of ipilimumab treated patients treated in CA184-004 and -007 at 3 time points. Week 0 (Baseline), 3 and 11. Plot shows anti-log RMA values at each time point.

FIG. 3B: Association of tumor gene expression of TAAs and ELISA titers of antibodies against these antigens. Gene expression was performed by Affymetrix gene expression chip and the numbers are the anti-log RMA values. The ELISA titers are OD₄₅₀ reading at dilution 1:400. Any OD₄₅₀ above (background+3×SD) is highlighted as a positive Ab response.

FIGS. 4A-F: Nucleotide and amino acid sequences of TAAs.

FIGS. 5A-B: ELISA assay on baseline (indicated as “week 1”) serum samples from CA184-004 (A) and -007 (B). OD₄₅₀ readings of serum dilution of 1:400 is displayed. Last row is the background+3×standard division (SD). Any OD₄₅₀ reading above this value was considered as positive.

FIGS. 6A-B: ELISA assay on week 11 serum samples from CA184-004 (A) and -007 (B). OD₄₅₀ readings of serum dilution of 1:400 is displayed. Last row is the background+3×standard division (SD). Any OD₄₅₀ reading above this value was considered as positive and are colored as a heat map.

FIG. 7: Table 5: Summary of ELISAs. ELISA assay on baseline serum samples from CA184-004 and -007. OD₄₅₀ readings of serum dilution of 1:400 is displayed. Last row is the background+3×standard division (SD). Any OD₄₅₀ reading above this value was considered as positive. Ratio is defined as the % positives survived >1 Y divided by % positive survived <1 Y.

FIG. 8: Table 6 shown in 6 panels with the last three panels consisting of the right side of the first three panels. The Table shows peripheral blood gene expression in patients treated with ipilimumab in CA184-004 and -007. The Table shows fold differences or changes in the gene expression from Week 0 (baseline) to week 3 and 11 in patients with CA or No-CA.

### DETAILED DESCRIPTION

The methods described herein are based at least on the discovery that subjects having advanced melanoma and being treated with ipilimumab survive longer than similar subjects if they have higher levels of antibodies to certain tumor associated antigens (TAAs). The antibodies specific for these TAAs may be used as biomarkers, e.g., prognostic, predictive biomarkers (such as markers of clinical efficacy) or biomarkers of clinical efficacy.

Provided herein are methods for treating a subject having cancer, e.g., melanoma. A method may comprise identifying a subject having cancer and having a level of antibodies to at least two TAAs that is higher than a predetermined antibody value for each of the two TAAs. A method may further comprise administering to the subject a therapeutically effective amount of a therapeutic for treating cancer.

Also provided herein are methods for treating a subject having cancer, e.g., melanoma, comprising (i) determining the level of antibodies to each of at least two TAAs in a subject having cancer; and (ii) administering to the subject a therapeutically effective dose of a therapeutic agent for treating the cancer if the level of antibodies specific for at least two TAAs in the subject is higher than a predetermined antibody value for each of the two TAAs.

Also provided herein are methods for determining whether a subject having cancer, e.g., melanoma, is likely to respond to a therapeutic agent for treating the cancer. A method may comprise determining the level of antibodies to each of at least two TAAs in a subject having cancer, wherein (i) a higher level of antibodies to each of at least two TAAs in the subject relative to a predetermined antibody value for each TAA indicates that the subject is likely to respond to a therapeutic agent for treating the cancer; and (ii) the absence of a higher level of antibodies to each of at least two TAAs in the subject relative to a predetermined antibody value for each TAA indicates that the subject is not likely to respond to a therapeutic agent for treating the cancer.

Also provided herein are methods for determining whether to treat a subject having cancer, e.g., melanoma, with a therapeutic agent for treating the cancer, comprising determining the level of antibodies to each of at least two TAAs in a subject having cancer, wherein (i) a higher level of antibodies to each of at least two TAAs relative to a predetermined antibody value for each of the TAAs indicates that the subject should be treated with a therapeutic agent for treating the cancer; and (ii) the absence of a higher level of antibodies to each of at least two TAAs in the subject relative to a predetermined antibody value for each TAA indicates that the subject should not be treated with a therapeutic agent for treating the cancer.

Also encompassed herein are methods for selecting subjects having cancer for treatment with a therapeutic for treating cancer. A method may comprise determining the level of antibodies to each of at least two TAAs in a subject having cancer, wherein (i) a higher level of antibodies to each of at least two TAAs relative to a predetermined antibody value for each of the TAAs indicates that the subject is a subject who should be treated with a therapeutic agent for treating the cancer; and (ii) the absence of a higher level of antibodies to each of at least two TAAs in the subject relative to a predetermined antibody value for each TAA indicates that the subject is not a subject who should not be treated with a therapeutic agent for treating the cancer.

Further provided herein are methods for predicting the length of survival of a subject having cancer, e.g., melanoma. A method may comprise determining the level of antibodies to each of at least two TAAs in a subject having cancer, wherein (i) a higher level of antibodies to each of at least two TAAs relative to a predetermined antibody value for each of the TAAs indicates that the subject is likely to have a longer survival relative to a subject who has cancer and does not have a level of antibodies to each of at least two TAAs that is higher than a predetermined antibody value for each TAA; and (ii) the absence of a higher level of antibodies to each of at least two TAAs in the subject relative to a predetermined antibody value for each TAA indicates that the subject is not likely to have a longer survival relative to a subject who has a level of antibodies to each of at least two TAAs that is higher than the predetermined antibody value for each TAA. A longer survival may be at least 200 days, 300 days, 400 days, 500 days, 600 days or more.

Further provided herein are methods for predicting the length of survival of a subject having cancer, e.g., melanoma, and being treated with a therapeutic agent for treating the cancer. A method may comprise determining the level of antibodies to each of at least two TAAs in a subject having cancer and being treated with a therapeutic agent for treating the cancer, wherein (i) a higher level of antibodies to each of at least two TAAs in the subject (e.g., measured prior to the beginning of the treatment with the therapeutic agent) relative to a predetermined antibody value for each of the TAAs indicates that the subject is likely to have a longer survival relative to a subject who has cancer and is treated with the therapeutic agent for treating the cancer and who does not have a level of antibodies to each of at least two TAAs (e.g., measured prior to the beginning of the treatment with the therapeutic agent) that is higher than the predetermined antibody value for each TAA; and (ii) the absence of a higher level of antibodies to each of at least two TAAs in the subject (e.g., measured prior to the beginning of the treatment with the therapeutic agent) relative to a predetermined antibody value for each TAA indicates that the subject is not likely to have a longer survival relative to a subject who has cancer and is treated with the therapeutic agent for treating the cancer and who has a level of antibodies to each of at least two TAAs (e.g., measured prior to the beginning of the treatment with the therapeutic agent) that is higher than the predetermined antibody value for each TAA.

Also provided are methods for determining the level of antibodies to each of at least two TAAs in a subject. A method may comprise providing a sample from a subject and measuring the level of antibodies to at least two TAAs.

Also encompassed herein are methods wherein, instead of measuring levels of antibodies to at least two TAA, the levels of expression of at least two TAAs is determined. A level of expression of a TAA may be determined in blood, e.g., whole blood, or in a tumor sample.

Measuring levels of antibodies to TAAs or levels of expression of TAAs may be conducted prior to the beginning of a therapy, e.g., a therapy with ipilimumab. Thus, methods may comprise measuring pre-existing (i.e., prior to initiation of therapy) levels of antibodies to at least two TAAs or pre-existing levels of expression of at least two TAAs. In certain embodiments, pre-existing levels of antibodies to at least two TAAs in a cancer patient (e.g., which are higher than predetermined antibody values for these TAAs) predicts that the cancer patient will respond to a treatment with an immunotherapeutic, e.g., ipilimumab, and/or survive longer relative to a subject who does not have pre-existing levels of antibodies to at least two TAAs (e.g., which are higher than predetermined antibody values for these TAAs).

A response to a therapeutic treatment, e.g., a treatment with ipilimumab, may be a clinical activity, such as a complete response to the therapeutic treatment, a partial response or stabilization of the disease. A response might also be a clinical benefit, such as tumor shrinkage, e.g., by at least 10%, 30%, 50%, 100% (2 fold), 3 fold, 5 fold, 10 fold or more, as determined, e.g., based on tumor weight or size.

### Tumor Associated Antigens (TAAs)

Determining the level of antibodies to at least two TAAs may comprise determining the level of antibodies to at least 2 TAAs selected from the group consisting of CTAG2, SSX2, SPANXA1, NLRP4, PBK and SPANXB1. Antibody levels may be determined separately for each TAA (or biomarker) or may be determined simultaneously (e.g., in one assay). Antibody levels to the following combinations of 2 TAAs may be determined. CTAG2 and SSX2; CTAG2 and SPANXA1; CTAG2 and NLRP4; CTAG2 and PBK; CTAG2 and SPANXB1; SSX2 and SPANXA1; SSX2 and NLRP4; SSX2 and PBK; SSX2 and SPANXB1; SPANXA1 and NLRP4; SPANXA1 and PBK; SPANXA1 and SPANXB1; NLRP4 and PBK; NLRP4 and SPANXB1; and PBK and SPANXB1 (see Table 1).

TABLE 1
Exemplary Combinations of 2 TAAs

| CTAG2 | SSX2 | SPANXA1 | NLRP4 | PBK | SPANXB1 |
|---|---|---|---|---|---|
| + | + | | | | |
| + | | + | | | |
| + | | | + | | |
| + | | | | + | |
| + | | | | | + |
| | + | + | | | |
| | + | | + | | |
| | + | | | + | |
| | + | | | | + |
| | | + | + | | |
| | | + | | + | |
| | | + | | | + |
| | | | + | + | |
| | | | + | | + |
| | | | | + | + |

In certain methods, antibody levels to at least 3, 4, 5 or more TAAs is determined Exemplary combinations of 3, 4 or 5 TAAs are shown in Tables 2, 3 and 4, respectively.

TABLE 2
Exemplary Combinations of 3 TAAs

| CTAG2 | SSX2 | SPANXA1 | NLRP4 | PBK | SPANXB1 |
|---|---|---|---|---|---|
| + | + | + | | | |
| + | + | | + | | |
| + | + | | | + | |
| + | + | | | | + |
| + | | + | + | | |
| + | | + | | + | |
| + | | + | | | + |
| + | | | + | + | |
| + | | | + | | + |
| + | | | | + | + |
| | + | + | + | | |
| | + | + | | + | |
| | + | + | | | + |
| | + | | + | + | |
| | + | | + | | + |
| | + | | | + | + |
| | | + | + | + | |
| | | + | + | | + |
| | | + | | + | + |
| | | | + | + | + |

TABLE 3
Exemplary Combinations of 4 TAAs

| CTAG2 | SSX2 | SPANXA1 | NLRP4 | PRK | SPANXB1 |
|---|---|---|---|---|---|
| + | + | + | + | | |
| + | + | + | | + | |
| + | + | + | | | + |
| + | | + | + | + | |
| + | | + | | + | + |
| + | | | + | + | + |
| | + | + | + | + | |
| | + | | + | + | + |
| | | + | + | + | + |

TABLE 4
Exemplary Combinations of 5 TAAs

| CTAG2 | SSX2 | SPANXA1 | NLRP4 | PBK | SPANXB1 |
|---|---|---|---|---|---|
| + | + | + | + | + | |
| + | | + | + | + | + |
| + | + | | + | + | + |
| + | + | + | | + | + |
| + | + | + | + | | + |
| | + | + | + | + | + |

In certain embodiments, the level of antibodies to CTAG2, SSX2, SPANXA1, NLRP4, PBK and SPANXB1 is determined. The level of antibodies to proteins other than CTAG2, SSX2, SPANXA1, NLRP4, PBK and SPANXB1 may also be determined. For example, in certain embodiments, the level of antibodies to NY-ESO-1 may be determined (Yuan et al., Proc. Natl. Acad. Sci., 108:16723 (2011)). In certain embodiments, the level of antibodies to any other antigen disclosed herein, e.g., in the Figures or Tables (e.g., BRAF) may be determined. The level of antibodies to CRISP3, GLUD1 and SOX2 may also be determined Generally, elevated levels of antibodies (e.g., at baseline or prior to treatment) to any two TAAs described herein may have a therapeutic, prognostic or predictive information, e.g., that a subject should or should not be treated with a therapeutic agent; that the subject is likely to respond to a therapeutic agent (e.g., by a reduction in tumor size); or that the subject is likely to live longer relative to a subject who does not have a higher level of at antibodies to at least two TAAs described herein. A higher level of antibodies to at least two TAAs described herein may also predict that the size of one or more tumors of the subject will shrink in size or weight (as determined, e.g., by measuring a radiological response, e.g., according to RECIST criteria), i.e., that the subject will have a medical benefit.

In certain embodiments, the level of antibodies to at least 3 TAAs selected from the group consisting of CTAG2, SSX2, SPANXA1, NLRP4, PBK and SPANXB1 is determined in a subject, and a higher level of antibodies to each of at least 2 TAAs from the at least 3 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of at least 2 TAAs from the at least 3 TAAs. In certain embodiments, the level of antibodies to at least 4 TAAs selected from the group consisting of CTAG2, SSX2, SPANXA1, NLRP4, PBK and SPANXB1 is determined in a subject, and a higher level of antibodies to each of at least 2 TAAs from the at least 4 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of at least 2 TAAs from the at least 4 TAAs. In certain embodiments, the level of antibodies to at least 5 TAAs selected from the group consisting of CTAG2, SSX2, SPANXA1, NLRP4, PBK and SPANXB1 is determined in a subject, and a higher level of antibodies to each of at least 2 TAAs from the at least 5 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of at least 2 TAAs from the at least 5 TAAs. In certain embodiments, the level of antibodies to CTAG2, SSX2, SPANXA1, NLRP4, PBK and SPANXB1 is determined in a subject, and a higher level of antibodies to each of at least 2 TAAs from the 6 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of at least 2 TAAs from the 6 TAAs.

In certain embodiments, the level of antibodies to at least 3 TAAs selected from the group consisting of CTAG2, SSX2, SPANXA1, NLRP4, PBK and SPANXB1 is determined in a subject, and a higher level of antibodies to each of at least 3 TAAs from the at least 3 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of at least 3 TAAs from the at least 3 TAAs. In certain embodiments, the level of antibodies to at least 4 TAAs selected from the group consisting of CTAG2, SSX2, SPANXA1, NLRP4, PBK and SPANXB1 is determined in a subject, and a higher level of antibodies to each of at least 3 TAAs from the at least 4 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of at least 3 TAAs from the at least 4 TAAs. In certain embodiments, the level of antibodies to at least 5 TAAs selected from the group consisting of CTAG2, SSX2, SPANXA1, NLRP4, PBK and SPANXB1 is determined in a subject, and a higher level of antibodies to each of at least 3 TAAs from the at least 5 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of at least 3 TAAs from the at least 5 TAAs. In certain embodiments, the level of antibodies to CTAG2, SSX2, SPANXA1, NLRP4, PBK and SPANXB1 is determined in a subject, and a higher level of antibodies to each of at least 3 TAAs from the 6 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of at least 3 TAAs from the 6 TAAs.

In certain embodiments, the level of antibodies to at least 4 TAAs selected from the group consisting of CTAG2, SSX2, SPANXA1, NLRP4, PBK and SPANXB1 is determined in a subject, and a higher level of antibodies to each of at least 4 TAAs from the at least 4 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of at least 4 TAAs from the at least 4 TAAs. In certain embodiments, the level of antibodies to at least 5 TAAs selected from the group consisting of CTAG2, SSX2, SPANXA1, NLRP4, PBK and SPANXB1 is determined in a subject, and a higher level of antibodies to each of at least 4 TAAs from the at least 5 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of at least 4 TAAs from the at least 5 TAAs. In certain embodiments, the level of antibodies to CTAG2, SSX2, SPANXA1, NLRP4, PBK and SPANXB1 is determined in a subject, and a higher level of antibodies to each of at least 4 TAAs from the 6 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of at least 4 TAAs from the 6 TAAs.

In certain embodiments, the level of antibodies to at least 5 TAAs selected from the group consisting of CTAG2, SSX2, SPANXA1, NLRP4, PBK and SPANXB1 is determined in a subject, and a higher level of antibodies to each of at least 5 TAAs from the at least 5 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of at least 5 TAAs from the at least 5 TAAs. In certain embodiments, the level of antibodies to each of CTAG2, SSX2, SPANXA1, NLRP4, PBK and SPANXB1 is determined in a subject, and a higher level of antibodies to each of at least 5 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of at least 5 TAAs from the 6 TAAs.

In certain embodiments, the level of antibodies to CTAG2, SSX2, SPANXA1, NLRP4, PBK and SPANXB1 is determined in a subject, and a higher level of antibodies to the 6 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to the 6 TAAs.

In certain embodiments, the level of antibodies to 3 TAAs selected from the group consisting of CTAG2, SSX2, SPANXA1, NLRP4, PBK and SPANXB1 is determined in a subject, and a higher level of antibodies to each of 2 TAAs from the 3 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of 2 TAAs from the 3

TAAs. In certain embodiments, the level of antibodies to 4 TAAs selected from the group consisting of CTAG2, SSX2, SPANXA1, NLRP4, PBK and SPANXB1 is determined in a subject, and a higher level of antibodies to each of 2 TAAs from the 4 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of 2 TAAs from the 4 TAAs. In certain embodiments, the level of antibodies to 5 TAAs selected from the group consisting of CTAG2, SSX2, SPANXA1, NLRP4, PBK and SPANXB1 is determined in a subject, and a higher level of antibodies to each of 2 TAAs from the 5 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of 2 TAAs from the 5 TAAs. In certain embodiments, the level of antibodies to CTAG2, SSX2, SPANXA1, NLRP4, PBK and SPANXB1 is determined in a subject, and a higher level of antibodies to each of 2 TAAs from the 6 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of 2 TAAs from the 6 TAAs.

In certain embodiments, the level of antibodies to 3 TAAs selected from the group consisting of CTAG2, SSX2, SPANXA1, NLRP4, PBK and SPANXB1 is determined in a subject, and a higher level of antibodies to each of 3 TAAs from the 3 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of 3 TAAs from the 3 TAAs. In certain embodiments, the level of antibodies to 4 TAAs selected from the group consisting of CTAG2, SSX2, SPANXA1, NLRP4, PBK and SPANXB1 is determined in a subject, and a higher level of antibodies to each of 3 TAAs from the 4 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of 3 TAAs from the 4 TAAs. In certain embodiments, the level of antibodies to 5 TAAs selected from the group consisting of CTAG2, SSX2, SPANXA1, NLRP4, PBK and SPANXB1 is determined in a subject, and a higher level of antibodies to each of 3 TAAs from the 5 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of 3 TAAs from the 5 TAAs. In certain embodiments, the level of antibodies to CTAG2, SSX2, SPANXA1, NLRP4, PBK and SPANXB1 is determined in a subject, and a higher level of antibodies to each of 3 TAAs from the 6 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of 3 TAAs from the 6 TAAs.

In certain embodiments, the level of antibodies to 4 TAAs selected from the group consisting of CTAG2, SSX2, SPANXA1, NLRP4, PBK and SPANXB1 is determined in a subject, and a higher level of antibodies to each of 4 TAAs from the 4 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of 4 TAAs from the 4 TAAs. In certain embodiments, the level of antibodies to 5 TAAs selected from the group consisting of CTAG2, SSX2, SPANXA1, NLRP4, PBK and SPANXB1 is determined in a subject, and a higher level of antibodies to each of 4 TAAs from the 5 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of 4 TAAs from the 5 TAAs. In certain embodiments, the level of antibodies to CTAG2, SSX2, SPANXA1, NLRP4, PBK and SPANXB1 is determined in a subject, and a higher level of antibodies to each of 4 TAAs from the 6 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of 4 TAAs from the 6 TAAs.

In certain embodiments, the level of antibodies to 5 TAAs selected from the group consisting of CTAG2, SSX2, SPANXA1, NLRP4, PBK and SPANXB1 is determined in a subject, and a higher level of antibodies to each of 5 TAAs from the 5 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of 5 TAAs from the 5 TAAs. In certain embodiments, the level of antibodies to each of CTAG2, SSX2, SPANXA1, NLRP4, PBK and SPANXB1 is determined in a subject, and a higher level of antibodies to each of 5 TAAs provides therapeutic, prognostic or predictive information, e.g., that a subject should or should not be treated with a therapeutic agent; that the subject is likely to respond to a therapeutic agent; or that the subject is likely to live longer relative to a subject who does not have a higher level of antibodies to each of 5 TAAs from the 6 TAAs.

A “level of antibodies” of a given TAA refers to the concentration of antibodies to a given TAA, e.g., in serum of a subject, or to a value that is derived from and is reflective of (or proportionate to) the concentration of antibodies to a given TAA, e.g., in serum of a subject. For example, when a level of antibodies is determined by ELISA, a level of antibodies may be defined as an optical density at 450 nm (OD₄₅₀). The OD₄₅₀ may result from an ELISA assay conducted on undiluted serum, or on serum that has been diluted, e.g., serum that has been diluted 10, 50, 100, 200, 300, 400 or 500 fold (i.e., dilutions 1:10; 1:50; 1:100; 1:200; 1:300, 1:400, and 1:500, respectively).

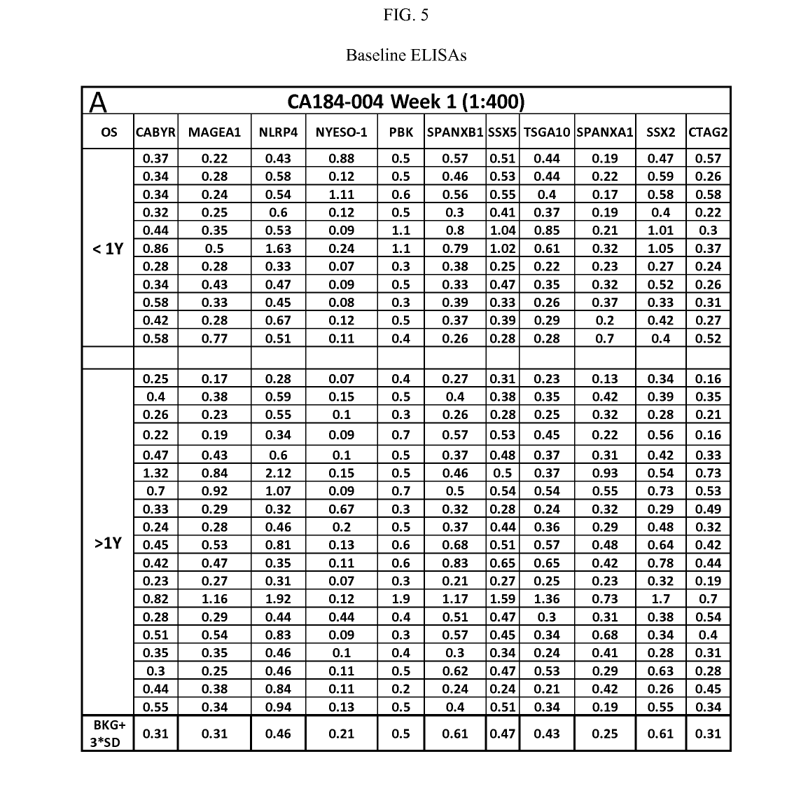

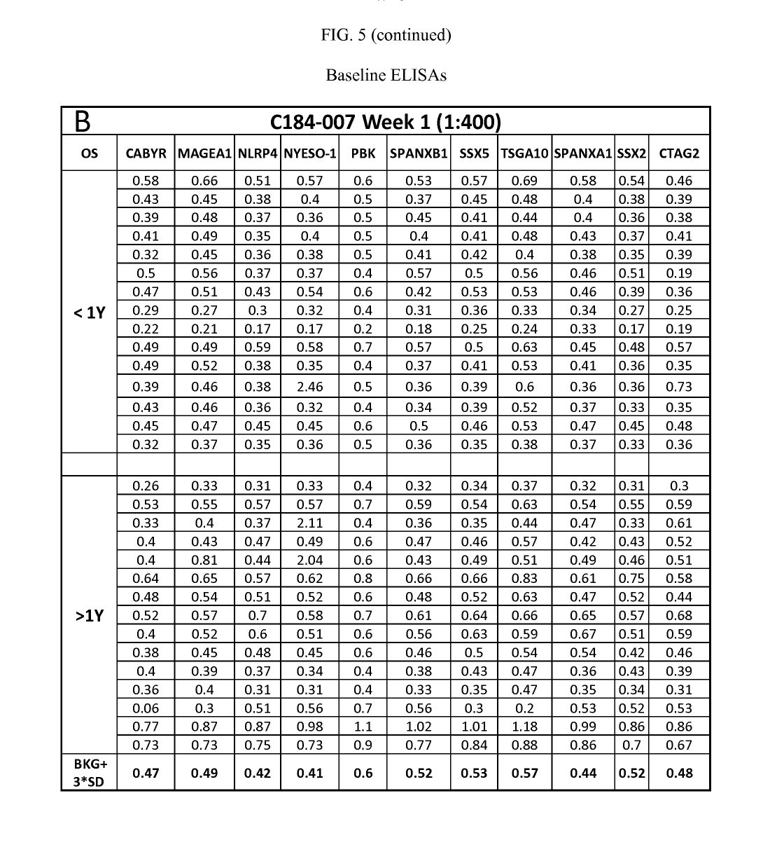

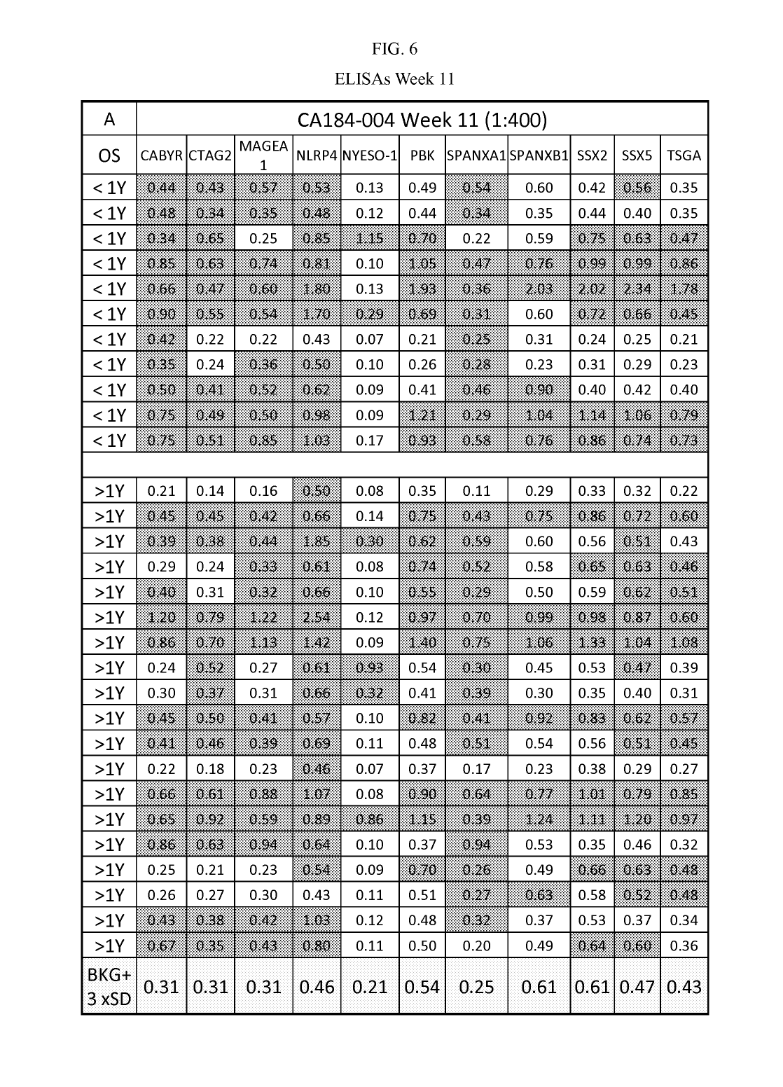

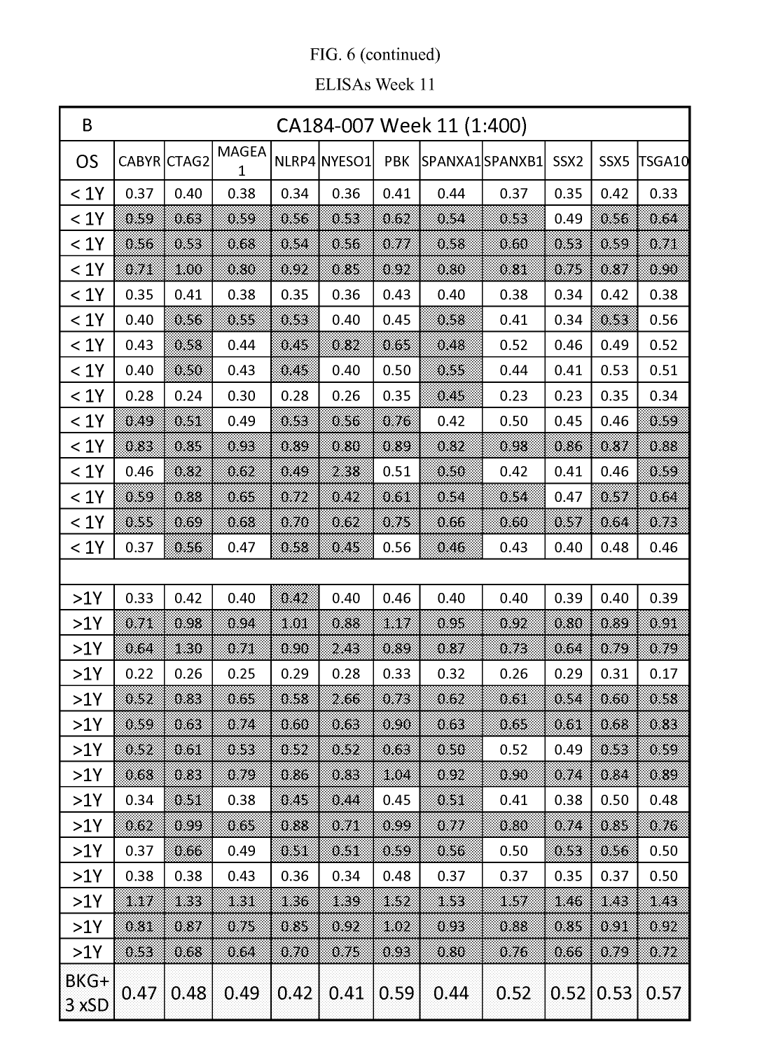

A “predetermined antibody value” for a given TAA is a level of antibody to the given TAA that is present in a control subject or is the average level of antibody to the given TAA present in at least 5, 10, 20, 50, 100, 1000 or more control subjects. A “control subject” is generally a subject who does not have an abnormally elevated level of antibodies to one or more TAAs. For example, a control subject may be a healthy subject, such as a subject who does not have a cancer or in whom a cancer is not detectable by standard means. In certain embodiments, a control subject is a subject who does not have melanoma, e.g., a subject who does not have advanced melanoma. A control subject may also be a subject who has a disease, e.g., cancer, but was later determined not to have an extended life or not to be responsive to a therapeutic agent due to the absence of elevated levels of one or more TAA. A predetermined antibody value for a given TAA may be the level of antibody to the TAA in one or more subjects (i.e., the concentration of antibodies or a value that is proportionate thereto), or it may be a value that is derived there from. In one embodiment, a predetermined antibody value for a given TAA is the addition of (1) the level of antibody to the TAA (e.g., an OD₄₅₀ at a particular dilution of serum) in control subjects and (2) 3 times the standard deviation (S.D.) between the level of antibody to the TAA in each of the control subjects. In one embodiment, a predetermined antibody value for a given TAA is the addition of (1) the level of antibody to the TAA (e.g., an OD₄₅₀ at a particular dilution of serum) in control subjects and (2) 2 times the standard deviation (S.D.) between the level of antibody to the TAA in each of the control subjects. In one embodiment, a predetermined antibody value for a given TAA is the addition of (1) the level of antibody to the TAA (e.g., an OD₄₅₀ at a particular dilution of serum) in control subjects and (2) 1 time the standard deviation (S.D.) between the level of antibody to the TAA in each of the control subjects. When comparing a level of antibodies to a given TAA in a subject to a predetermined antibody value for the TAA, the level of antibodies and the level of antibodies that forms the basis of the predetermined antibody value must have been determined by the same method. For example, when the level of antibodies to a given TAA is expressed as the OD₄₅₀ of a 1:100 dilution of a subject's serum, then that value should be compared to a predetermined antibody value obtained from a 1:100 dilution of the serum of the control subject(s). In one embodiment, a level of antibodies to a given TAA in a subject is obtained by determining the OD₄₅₀ value by ELISA of a 1:400 dilution of serum of the subject. The value obtained may be compared to a predetermined antibody value that corresponds to the addition of (1) the average OD₄₅₀ of a 1:400 dilution of sera from at least 10, 50, 100, 500, or 1000 control subjects; and (2) 3 times the S.D. of the OD₄₅₀ values of the healthy subjects. A higher level of antibodies in the subject relative to the predetermined antibody value is indicative of a therapeutic, prognosis or prediction, as further described herein. Exemplary predetermined antibody values for certain TAAs are provided in the Examples (last line of the Tables in FIGS. 5 and 6) and are reproduced below in Table 4:

TABLE 4
Exemplary Predetermined Antibody Values for Given TAAs

| | Predetermined |
|---|---|
| Name of TAA | Antibody Value* |
| CTAG2 | 0.31 |
| SSX2 | 0.61 |
| SPANXA1 | 0.25 |
| NLRP4 | 0.46 |
| PBK | 0.5 |
| SPANXB1 | 0.61 |

*As determined from the addition of (1) the average OD₄₅₀ of a 1:400 dilution of sera from subjects having low levels of antibodies to a given TAA; and (2) 3 times the S.D. of the OD₄₅₀ values of the subjects.

Generally, the following ranges of values may be used as predetermined antibody values for each of the following TAAs (based on OD₄₅₀ values obtained in ELISAs of 1:400 diluted serum): a predetermined antibody value for CTAG2 may be a value within one of the following ranges: 0.2 to 0.5; 0.25 to 0.4; and 0.3 to 0.35. A predetermined antibody value for SSX2 may be a value within one of the following ranges: 0.5 to 0.8; 0.55 to 0.7; and 0.6 to 0.65. A predetermined antibody value for SPANXA1 may be a value within one of the following ranges: 0.1 to 0.4; 0.2 to 0.3 or 0.22 to 0.27. A predetermined antibody value for NLRP4 may be a value within one of the following ranges: 0.3 to 0.6; 0.4 to 0.55 and 0.4 to 0.5. A predetermined antibody value for PBK may be a value within one of the following ranges: 0.3 to 0.7; 0.4 to 0.6 and 0.45 to 0.55. A predetermined antibody value for SPANXB1 may be a value within one of the following ranges: 0.5 to 0.8; 0.55 to 0.7; and 0.6 to 0.65.

The phrase “a higher level of antibodies relative to a predetermined antibody value” refers to a level of antibodies that is at least 1%, 5%, 10%, 30%, 50%, 70%, 100% (2 fold), 3 fold, 5 fold, 10 fold, 30 fold, 50 fold, 100 fold or higher than the predetermined antibody value. When a predetermined antibody value is a concentration of antibody (i.e., without having added “3×SD”), then the phrase “a higher level of antibodies relative to a predetermined antibody value” may refer to a level of antibodies that is preferably at least 50%, 100% (2 fold), 3 fold, 5 fold, 10 fold, 30 fold, 50 fold, 100 fold or higher than the predetermined antibody value.

The following is a brief description of preferred TAAs:

CTAG2 is also referred to as “cancer/testis antigen 2”; ESO2; CAMEL; CT6.2; CT6.2a; CT6.2b; LAGE-1; and LAGE2B. CTAG2 is a tumor antigen that belongs to the ESO/LAGE family of cancer-testis antigens and is expressed in a wide array of cancers including melanoma, breast cancer, bladder cancer and prostate cancer. Human CTAG2 exists as two isoforms: 1) cancer/testis antigen 2 isoform LAGE-1a having the amino acid sequence set forth as GENBANK® Accession No. NP_{—}758965.1 and encoded by the nucleotide sequence set forth as GENBANK® Accession No. NM_{—}172377.3; and 2) cancer/testis antigen 2 isoform LAGE-1b having the amino acid sequence set forth as GENBANK® Accession No. NP_{—}066274.1 and encoded by the nucleotide sequence set forth as GENBANK® Accession No. NM_{—}020994.3. They both have been assigned Gene ID: 30848. The level of antibodies against either one of these isoforms can be measured in the methods described herein. In certain embodiments, the level of antibodies to the protein encoded by the nucleotide sequence having GENBANK® Accession No. NM_{—}172377.3 and/or NM_{—}020994.3 is measured. In certain embodiments, the level of antibodies to LAGE-1 is measured. In certain embodiments, the level of antibodies to LAGE-2 is measured. In certain embodiments, the level of antibodies to LAGE-1 and to LAGE-2 is measured.

SSX2 is also referred to as “synovial sarcoma, X breakpoint 2”; SSX; HD21; SSX2B; CT5.2a; and HOM-MEL-40. SSX2 belongs to the family of highly homologous synovial sarcoma X (SSX) breakpoint proteins and are capable of eliciting spontaneously humoral and cellular immune responses in cancer patients. Human SSX2 exists as two isoforms: 1) SSX2 isoform a having the amino acid sequence set forth as GENBANK® Accession No. NP_{—}003138.3 and encoded by the nucleotide sequence set forth as GENBANK® Accession No. NM_{—}003147.4; and 2) SSX2 isoform b having the amino acid sequence set forth as GENBANK® Accession No. NP_{—}783629.1 and encoded by the nucleotide sequence set forth as GENBANK® Accession No. NM_{—}175698.1. They both have been assigned Gene ID: 6757. In certain embodiments, the level of antibodies to the protein encoded by the nucleotide sequence having GENBANK® Accession No. NM_{—}003147.4 and/or NM_{—}175698.1 is measured. In certain embodiments, the level of antibodies to SSX2 isoform a is measured. In certain embodiments, the level of antibodies to SSX2 isoform b is measured. In certain embodiments, the level of antibodies to SSX2 isoforms a and b is measured.

SPANXA1 is also referred to as “sperm protein associated with the nucleus, X-linked, family member A1”; NAP-X; SPANX; CT11.1; SPANXC; SPANXD; SPAN-Xa; SPAN-Xb; SPANX-C; SPANX-D; SPANXA2; SPANX-A2. SPANXA1 is a member of the SPANX family of cancer/testis-associated genes. Human SPANXA1 has the amino acid sequence set forth as GENBANK® Accession No. NP_{—}038481.2 and is encoded by the nucleotide sequence set forth as GENBANK® Accession No. NM_{—}013453.2. SPANXA1 has been assigned Gene ID: 30014.

NLRP4 is also referred to as “NLR family, pyrin domain containing 4”; “NACHT, LRR and PYD domains-containing protein 4”; CT58; PAN2; RNH2; NALP4; PYPAF4; and CLR19.5. NALPs are implicated in the activation of proinflammatory caspases. Human NLRP4 has the amino acid sequence set forth as GENBANK® Accession No. NP_{—}604393.2 and is encoded by the nucleotide sequence set forth as GENBANK® Accession No. NM_{—}134444.4. NLRP4 has been assigned Gene ID: 147945.

PBK is also referred to as “PDZ binding kinase”; “lymphokine-activated killer T-cell-originated protein kinase”; SPK; CT84; TOPK; and Nori-3. PBK is a serine/threonine kinase related to the dual specific mitogen-activated protein kinase kinase (MAPKK) family. Human PBK has the amino acid sequence set forth as GENBANK® Accession No. NP_{—}060962.2 and is encoded by the nucleotide sequence set forth as GENBANK® Accession No. NM_{—}018492.2. PBK has been assigned Gene ID: 55872.

SPANXB1 is also referred to as “SPANX family, member B1”; “sperm protein associated with the nucleus on the X chromosome B/F”; B1; CT11.2; SPANXB; SPANX-B; SPANXB2; and SPANXF1. SPANXB1 is a member of the SPANX family of cancer/testis-associated genes. Human SPANXB1 has the amino acid sequence set forth as GENBANK® Accession No. NP_{—}115850.1 and is encoded by the nucleotide sequence set forth as GENBANK® Accession No. 1.NM_{—}032461.2.

Levels of antibodies may be determined in a blood, plasma or serum sample of a subject. Accordingly, certain methods comprise first obtaining a serum sample from a subject. Serum samples may be obtained according to methods known in the art. Levels of antibodies in serum samples may be determined by ELISA, e.g., standard ELISA, such as described in the Examples. Proteins that may be used in the ELISA assays include full length proteins or antigenic fragments thereof, as further described herein.

Levels of antibodies in serum samples may be also be determined by multiplex technologies, e.g., Luminex Proteins that may be used in the multiplex assays include full length proteins or antigenic fragments thereof, as further described herein.

In certain embodiments, a method comprises obtaining a serum sample from a subject, diluting the serum, e.g., 1:400, conducting an ELISA assay using at least two TAAs selected from the group consisting of CTAG2, SSX2, SPANXA1, NLRP4, PBK and SPANXB 1 and obtaining an OD₄₅₀ for each of at least two TAAs. The OD₄₅₀ values obtained for each of the TAAs are then compared to the predetermined antibody values for each of these two TAAs.

### Diseases

The methods described herein relate to prognostic and predictive methods as well as therapeutic methods for subjects having a disease or disorder, e.g., cancer. For example, methods for treating a subject having cancer are provided. Other methods include methods for determining whether a subject having cancer is likely to respond to a therapeutic treatment for the cancer; methods for determining whether to treat a subject having cancer; and methods for determining or predicting the length of survival of a subject having cancer.

The methods apply to cancer, such as advanced cancer and metastatic cancer. Exemplary cancers include malignant melanoma, such as subjects having stage I, stage II, stage III or stage IV melanoma, e.g., as determined by histologic or cytologic diagnosis of malignant melanoma. Thus, the methods may be prognostic or predictive of survival or response to treatment in subjects having advanced melanoma, such as metastatic melanoma, e.g., stage III or IV melanoma, such as unresectable stage III and IV melanoma. The methods described herein may also be applied to subjects having any of the following types of cancers: lung cancer, non-small cell lung cancer, small cell lung cancer, prostate cancer, glioma, gastrointestinal cancer, renal cancer, ovarian cancer, liver cancer, colorectal cancer, endometrial cancer, kidney cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, bone cancer, bone tumors, adult malignant fibrous histiocytoma of bone; childhood malignant fibrous histiocytoma of bone, sarcoma, pediatric sarcoma, sinonasal natural killer, neoplasms, plasma cell neoplasm; myelodysplastic syndromes; neuroblastoma; testicular germ cell tumor, intraocular melanoma, myelodysplastic syndromes; myelodysplastic/myeloproliferative diseases, synovial sarcoma, chronic myeloid leukemia, acute lymphoblastic leukemia, Philadelphia chromosome positive acute lymphoblastic leukemia (Ph+ ALL), multiple myeloma, acute myelogenous leukemia, chronic lymphocytic leukemia, mastocytosis and any symptom associated with mastocytosis, and any metastasis thereof. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, mast cell leukemia, extensive stage small cell lung cancer, early stage/resectable breast cancer, stage III-IV or recurrent pancreatic cancer that cannot be removed by surgery, locally advanced, unresectable or metastatic pancreatic cancer, urothelial carcinoma undergoing surgical resection, metastatic urothelial carcinoma, hormone-refractory prostate cancer, non small cell lung cancer (nsclc) or small cell lung cancer (sclc), non-squamous non-small cell lung cancer, advanced or refractory solid tumors, recurrent or refractory lymphoma, metastatic renal cell cancer, ovarian epithelial cancer, melanoma, acute myeloid leukemia, myelodysplastic syndrome, or non-small cell lung cancer, advanced synovial sarcoma, cancers listed elsewhere in here, in addition to other cancers.

### Therapeutic Agents

Certain methods described herein provide for treatment of a subject having a disease, e.g., cancer, with a therapeutic agent. For example, certain methods described herein are methods for treating cancer with a therapeutic agent for cancer. A therapeutic agent for treating cancer may be an immunotherapeutic agent, such as an agent that stimulates immune responses. Exemplary immunotherapeutic agents that may be used include those that stimulate an immune response (or the immune system or part thereof) by modulating the co-stimulatory pathway, e.g., the pathways that involves the B-7 family of molecules and/or the CD28 and CTLA-4 family of molecules.

Exemplary immunotherapeutic agents are CTLA-4 antagonists. Suitable anti-CTLA-4 antagonist agents for use in the methods described herein, include, without limitation, anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, anti-CTLA-4 molecules based on fibronectin, e.g., ADNECTINS™, anti-CTLA-4 domain antibodies, single chain anti-CTLA-4 fragments, heavy chain anti-CTLA-4 fragments, light chain anti-CTLA-4 fragments, modulators of the co-stimulatory pathway, MDX-010 (ipilimumab), tremelimumab, the antibodies disclosed in PCT Publication No. WO 2001/014424, the antibodies disclosed in PCT Publication No. WO 2004/035607, the antibodies disclosed in U.S. Published Application No. 2005/0201994, and the antibodies disclosed in granted European Patent No. EP 1212422 B1. Additional CTLA-4 antibodies that may be used are described in U.S. Pat. Nos. 5,811,097, 5,855,887, 6,051,227 and 6,984,720; in PCT Publication Nos. WO 01/14424 and WO 00/37504; and in U.S. Publication Nos. 2002/0039581 and 2002/086014. Other anti-CTLA-4 antibodies that can be used in a method described herein include, for example, those disclosed in: WO 98/42752; U.S. Pat. Nos. 6,682,736 and 6,207,156; Hurwitz et al., Proc. Nall. Acad. Sci. USA, 95(17):10067-10071 (1998); Camacho et al., J. Clin. Oncology, 22(145), Abstract No. 2505 (2004) (antibody CP-675206); Mokyr et al., Cancer Res., 58:5301-5304 (1998), U.S. Pat. Nos. 5,977,318, 6,682,736, 7,109,003, 7,132,281, 7,452,535, 7,465,446, 7,744,875, 7,605,238 and 8,017,114; EP Patent Nos. 1503794, 0865293 B1, 1137436 B1, and 0606217 B2. Each of these references is specifically incorporated herein by reference for purposes of description of CTLA-4 antibodies.

A preferred clinical CTLA-4 antibody is human monoclonal antibody 10D1 (also referred to as MDX-010 and ipilimumab and available from Bristol-Myers Squibb Company), disclosed in WO 01/14424. As is known in the art, ipilimumab refers to an anti-CTLA-4 antibody, and is a fully human IgG₁ antibody derived from transgenic mice having human genes encoding heavy and light chains to generate a functional human repertoire. Ipilimumab can also be referred to by its CAS Registry No. 477202-00-9, and is disclosed as antibody 10D1 in PCT Publication No. WO 01/14424; U.S. Pat. Nos. 6,984,720, 7,605,238 and 8,017,114; and EP Patent No. 1212422 B1, all of which are incorporated herein by reference in their entirety and for all purposes. Ipilimumab is a human monoclonal antibody that specifically binds to CTLA-4, and comprises a light chain variable region having SEQ ID NO: 1 and a heavy chain variable region having SEQ ID NO: 2. Pharmaceutical compositions of ipilimumab include all pharmaceutically acceptable compositions comprising ipilimumab and one or more diluents, vehicles and/or excipients. Examples of a pharmaceutical composition comprising ipilimumab are provided in PCT Publication No. WO 2007/67959.

Light chain variable region of ipilimumab:

(SEQ ID NO: 1)
EIVLTQSPGTLSLSPGERATLSCRASQSVGSSYLAWYQQKPGQA
PRLLIYGAFSRATGIPDRFSGSGSGTDFTLTISRLEPEDFAVYY
CQQYGSSPWTFGQGTKVEIK

Heavy chain variable region of ipilimumab:

(SEQ ID NO: 2)
QVQLVESGGGVVQPGRSLRLSCAASGFTFSSYTMHWVRQAPGKG
LEWVTFISYDGNNKYYADSVKGRFTISRDNSKNTLYLQMNSLRA
EDTAIYYCARTGWLGPFDYWGQGTLVTVSS

### Measuring Levels of TAAs

In certain embodiments, a therapeutic, prognostic or predictive method comprises determining the level of at least one or two TAAs in addition to or as a substitution to determining the level of antibodies to one or more TAAs. Levels of TAAs may be determined by first obtaining a sample of a tumor from the subject and determining the level of TAA in the sample. The level of TAA may be determined by measuring the protein level or mRNA level. Any of the methods known in the art for measuring protein or mRNA may be used. The level of TAA may then be compared to a predetermined antibody value for the TAA, which predetermined antibody value may be the level of TAA that is present in one or more control subjects.

### Kits

Also provided herein are kits, e.g., kits for use in the methods described herein. A kit may comprise one or more reagents for determining the level of antibodies to 1, 2, 3, 4, 5, 6 or more TAAs, e.g., selected from the group consisting of CTAG2, SSX2, SPANXA1, NLRP4, PBK and SPANXB1. For example, a kit may comprise 1, 2, 3, 4, 5, 6, or more isolated TAAs or antigenic portions thereof for use in an assay for determining the level of antibodies to these TAAs, such as an ELISA. A kit may comprise 1, 2, 3, 4, 5, 6, or more TAAs or antigenic portions thereof for use in a multiplex assay for determining the level of antibodies to these TAAs, such as Luminex based technology. Provided herein are arrays comprising 1, 2, 3, 4, 5, 6, or more TAAs or antigenic portions thereof. In some embodiments, arrays do not comprise any other proteins or portions thereof, other than proteins that may be useful for as controls.

A kit may comprise, e.g., one or more container means such as vials, tubes, and the like, each of the container means comprising one of the separate elements to be used in the method. For example, one of the container means can comprise a means for measuring the level of antibodies to one or more TAAs in a patient sample and/or instructions for interpreting the measurement value obtained. Another example of a container means can comprise one or more vials containing a pharmaceutically acceptable amount of a therapeutic agent.

The kit of the invention will typically comprise the container described above and one or more other containers comprising materials desirable from a commercial and user standpoint, including buffers, diluents, filters, needles, syringes, and package inserts with instructions for use. The kit may also comprise, for example, a means for obtaining a biological sample from an individual. Means for obtaining biological samples from individuals are well known in the art, e.g., catheters, syringes, and the like, and are not discussed herein in detail. A label can be present on the container to indicate that the composition is used for a specific therapy or non-therapeutic application, and can also indicate directions for either in vivo or in vitro use.

Kits useful in practicing therapeutic methods disclosed herein can also contain a therapeutic compound, e.g., an immunotherapeutic compound. Specifically contemplated by the invention is a kit comprising an anti-CTLA-4 antibody, either alone or in combination with another immunotherapy agent, such as a tubulin stabilizing agent (e.g., pacitaxol, epothilone, taxane, etc.); a peptide vaccine such as PROVENGE®; and/or a second co-stimulatory pathway modulator, such as, tremelimumab.

In addition, the kits can include instructional materials containing directions (i.e., protocols) for the practice of the methods described herein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips, and the like), optical media (e.g., CD ROM), and the like. Such media can include addresses to internet sites that provide such instructional materials.

The following representative Examples contain important additional information, exemplification and guidance which can be adapted to the practice of this invention in its various embodiments and the equivalents thereof. These examples are intended to help illustrate the invention, and are not intended to, nor should they be construed to, limit its scope.

### EXAMPLES

### Example 1

### Anti-Tumor Antibody Response Profiling of Ipilimumab Treated Patients: Potential Predictive and Pharmacodynamic Biomarkers of Response and Survival

### Introduction

The immune system can fail to combat cancer when tumors develop mechanisms to evade or suppress these defenses. Several drugs have been designed to enhance immune reactivity to cancer by blocking immuno-regulatory proteins on the immune cells, such as cytotoxic T lymphocyte antigen-4 (CTLA4) and (programmed death 1) PD-1. Ipilimumab is one such agent which recognizes and blocks CTLA-4, an immune suppressor protein, upregulated and expressed on a subset of T cells upon their activation (1, 2). The end result of this blockade is sustained activation and proliferation state of CD4⁺ and CD8⁺ T cells (1). In several phase II and two controlled phase III clinical trials, ipilimumab proved to prolong survival in metastatic melanoma patients (3, 4). Based on these results, ipilimumab has been approved by the health authorities for the treatment of advanced metastatic melanomas.

Treatment with ipilimumab however is accompanied by a number of immune-mediated adverse reactions that in some cases might be severe (5). Thus, identification of biomarkers which would predict response or prolonged survival after treatment with this agent might have a significant impact on patient care. In a previous report, gene expression profiling of metastatic melanomas indicated that in addition to an increase in cytotoxic T cell markers, treatment with ipilimumab caused an increase in the expression of a number of immunoglobulin (Ig) genes in the tumors (6). However, the specificity of these Ig genes could not be established from such analysis. Other investigators have reported Ab responses toward a limited number of tumor associated antigens (TAAs) and associations with clinical response to ipilimumab have also been observed (7-9). Thereby, Ab responses to TAAs including melanoma associated antigens (MAAs) might have potential value as pharmacodynamic (PD) or predictive biomarkers of response and/or survival in ipilimumab treated patients. The lack of high sensitivity and specificity of the previously explored anti-tumor antibodies prompted us to use a broader screening method to identify novel anti-melanoma Abs as biomarkers of ipilimumab efficacy. Retrospective analysis of sera from a phase II clinical trial was performed for screening immune responses toward 30 known TAAs using an Ab array platform. Pre-existing Ab responses toward a number of these TAAs appeared to be predictive of long survival in these patients. Selected antibodies were further confirmed by ELISA in the same sample set. Additionally, these findings were confirmed in an independent set of sera from another phase II ipilimumab trial in metastatic melanoma patients. Treatment with ipilimumab also resulted in the expansion of Ab responses toward a broader range of TAAs. Together, these data suggest that humoral responses toward TAAs might have potential value as biomarkers of clinical efficacy for ipilimumab.

### Materials and Methods

Study Design. The multicenter, phase II clinical trial CA184-004 enrolled 82 previously-treated and untreated patients with unresectable stage III or IV melanoma, randomized 1:1 into 2 arms to receive up to 4 intravenous infusions of either 3 or 10 mg/kg ipilimumab every 3 weeks (Q3W) in the induction phase. In CA184-007 trial, treatment-naïve or previously treated patients with unresectable stage III/IV melanoma (N=115) received open-label ipilimumab (10 mg/kg every 3 wks for four doses) and were randomized to receive concomitant blinded prophylactic oral budesonide (9 mg/d with gradual taper through week 16) or placebo (4). Complete study design, patient characteristics and endpoint reports of these trials have been described elsewhere (10, 11). Both studies were conducted in accordance with the ethical principles originating from the current Declaration of Helsinki and consistent with International Conference on Harmonization Good Clinical Practice and the ethical principles underlying European Union Directive 2001/20/EC and the United States Code of Federal Regulations, Title 21, Part 50 (21 C.F.R. 50). The protocols and patient informed consent forms received appropriate approval by all Institutional Review Boards or Independent Ethics Committees prior to study initiation. All participating patients (or their legally acceptable representatives) gave written informed consent for these biomarker focused studies.

Affymetrix Gene Expression Analysis. Whole blood mRNA was available from both studies at three time points (Baseline, Week 3 and Week 11) from most patients. Also tumor mRNA samples were available from 80 out of 82 patients in CA184-004, from which 24 samples had matching sera from the patients included in these analyses. Gene expression profiling was performed by methods described previously (6).

Serum Samples. In CA184-004 and CA184-007 trials, metastatic melanoma patients received 4 doses of ipilimumab with three weeks interval between each dose. Paired serum samples collected at baseline and week 11 post-treatment (2 weeks after the last dose of ipilimumab) from 30 ipilimumab-treated patients in CA184-004 trial were used as the training set. Serum samples from CA184-007 collected at similar time points as CA184-004 were used as an independent data set for the confirmation of the findings in the first experiment.

Array Analysis. Sera were diluted 1:100 in sample buffer and exposed to a protein microarray (Serametrix, Encinitas, Calif.) containing 37 full-length human tumor antigens immobilized onto a planar solid support medium. After 3×15 minutes wash with wash buffer, the arrays were probed with the secondary Ab, mouse anti-human-IgG Cy-5 conjugate (1:10,000). The array was then read at 670 nm using a microarray scanner (Molecular Devices, Sunnyvale, Calif.) and data recorded as Relative Fluorescent Units (RFU). Data were distributed into intervals corresponding to High (score 3), Medium (Score 2) and Low (score 1) or no-detectable (score 0) fluorescence based on multiples of background signal. Signals greater than 3,000 RFU were reported as positive. For illustration and analysis purposes, relative sum of the array scores [(sum of scores in the cohort/number of patients)×100] was used to delineate differences between patient cohorts in the analysis.

Enzyme-linked Immunosorbent Assay (ELISA). Based on the preliminary screening results from the array analysis, a panel of 11 potential antigens (CABYR, CTAG2, MAGEA1, NLRP4, NYESO1, PBK, SPANXA1, SPANXB1, SSX2, SSX5, TSGA10) was selected for further confirmation of the array findings by ELISA which is a more quantitative assay. Sera were tested by standard ELISA using serial dilutions in duplicate wells of 96 well plates coated with full-length human recombinant proteins corresponding to the selected antigens. Bound antibodies were detected using a secondary Ab conjugate and subsequent detection by a colorimetric reaction. Plates were read using a standard ELISA plate reader (Molecular Devices) at 450 nm (OD₄₅₀). For each dilution, the average of the duplicate data was calculated across all dilutions. Seropositive patients were defined as those with an OD₄₅₀ reading above (mean background+3×standard Division) at the dilution of 1:400 of the sera.

Statistical Analysis. Gene expression analyses were performed by methods described previously (6). For survival analysis Kaplan-Meier estimates of overall survival were computed. Log-rank (Mantel-Cox) Test was used to establish association of Ab responses with overall survival. Patients were divided into two groups: those with Ab responses toward 0-1 selected antigens or those with response to 2-3 antigens.

### Results

### Identification of Potential Predictive-Prognostic Anti-Tumor Ab Responses in Ipilimumab Treated Patients by Ab Array

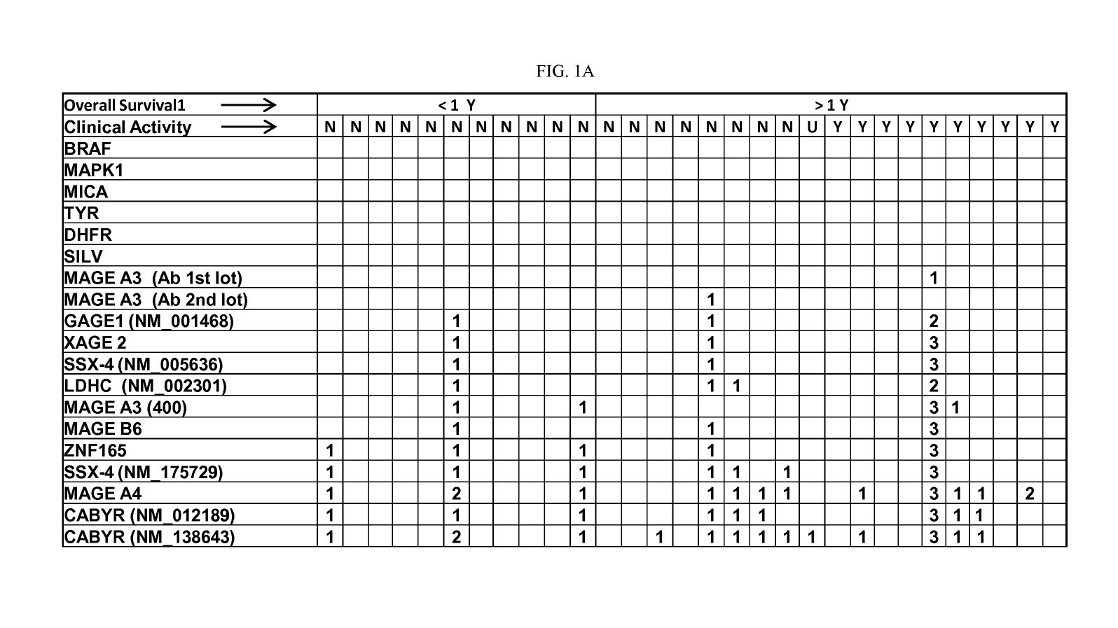

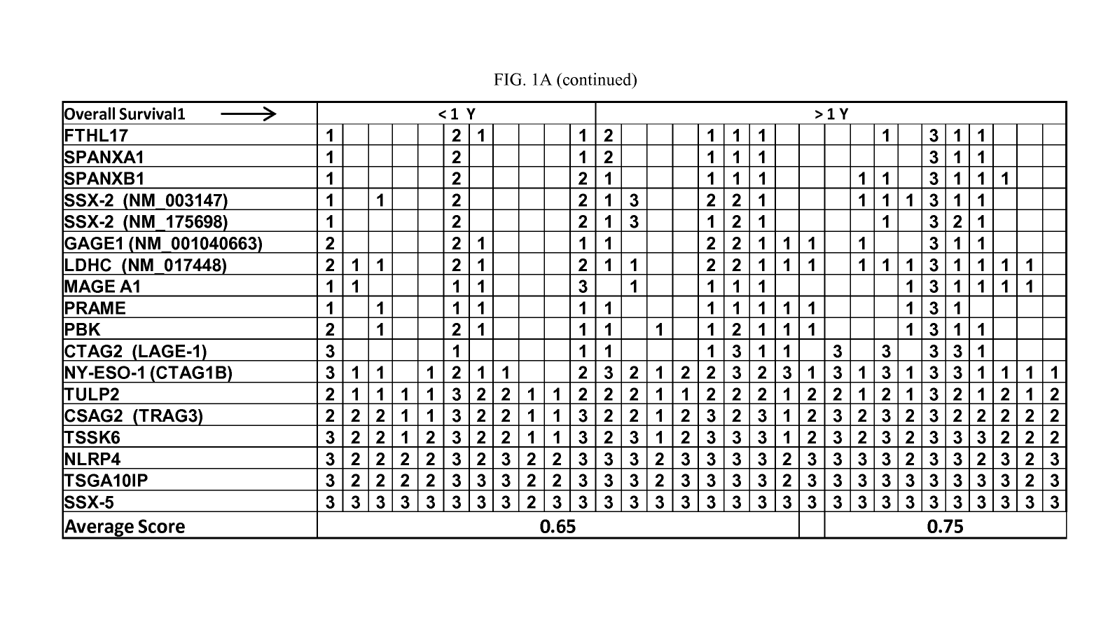

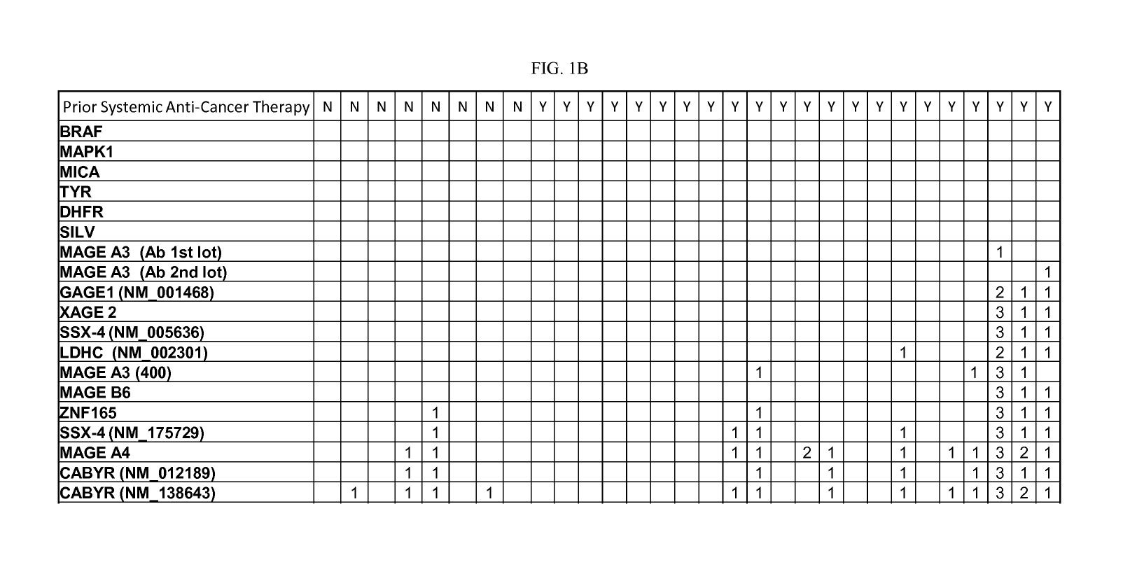

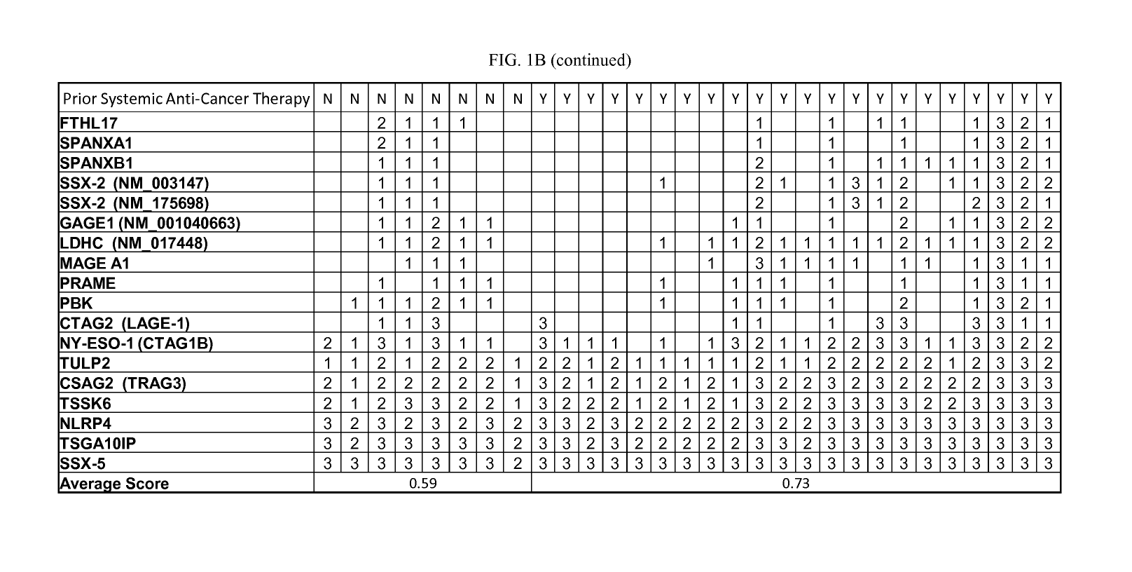

Baseline serum samples from 30 patients treated with ipilimumab were tested for the presence of preexisting Ab responses toward a panel of 30 known TAAs. Out of the 30 patients, 10 patients showed clinical activity (CA, based on mWHO criteria), 19 had No-CA and 1 patient was classified as unknown. An apparent trend toward higher and broader Ab responses was detected for patients with CA as compared to those without. Although the array does not provide a quantitative measure of Ab responses, 4 different levels of signal intensity (scale of 0-3) could be detected, with 0 no-detectable signal and 3 the highest RFU. According to this scoring system, patients with CA had slightly greater Ab responses toward TAAs, than those patients with No-CA (average score for all measured analytes=0.75 vs. 0.65, respectively) (FIG. 1A).

Twenty two out of 30 patients had received previous systemic anti-cancer therapy and 8 did not. Based on array data, patients who had received previous systemic anti-cancer therapy had apparently greater Ab responses than patients who were naïve for previous systemic therapy (average score: 0.59 vs. 0.73, respectively). These patients also had Ab responses toward a broader range of TAAs (FIG. 1B).

### Confirmation of Potential Predictive-Prognostic Ab Biomarkers by ELISA

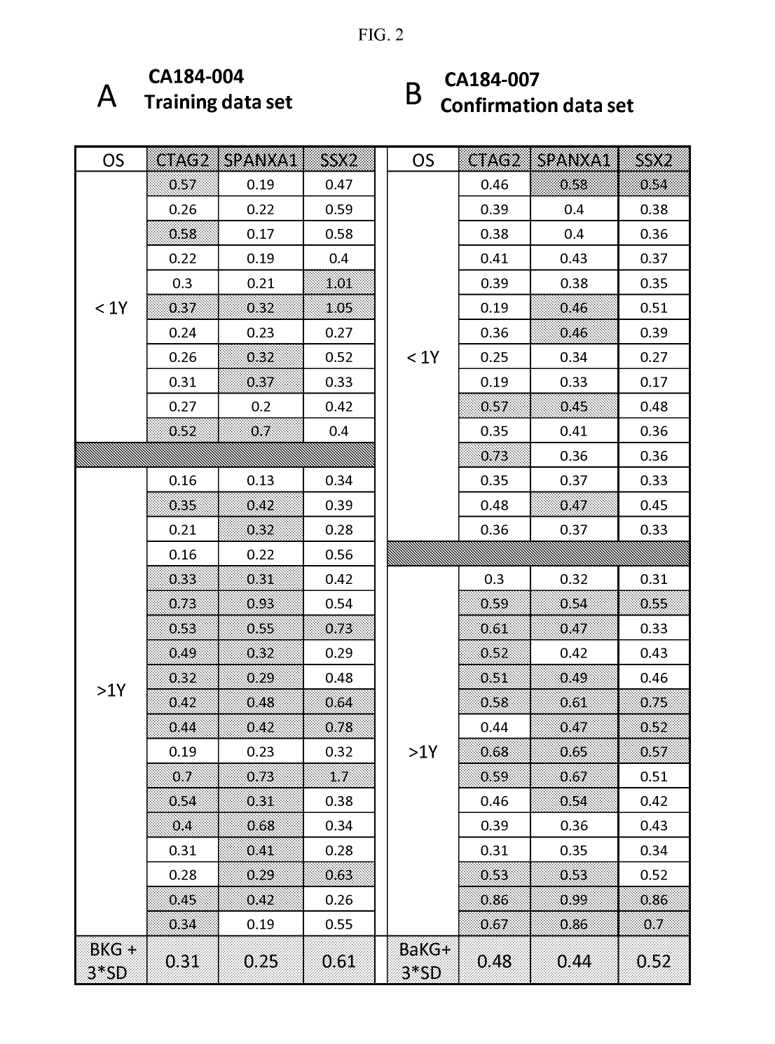

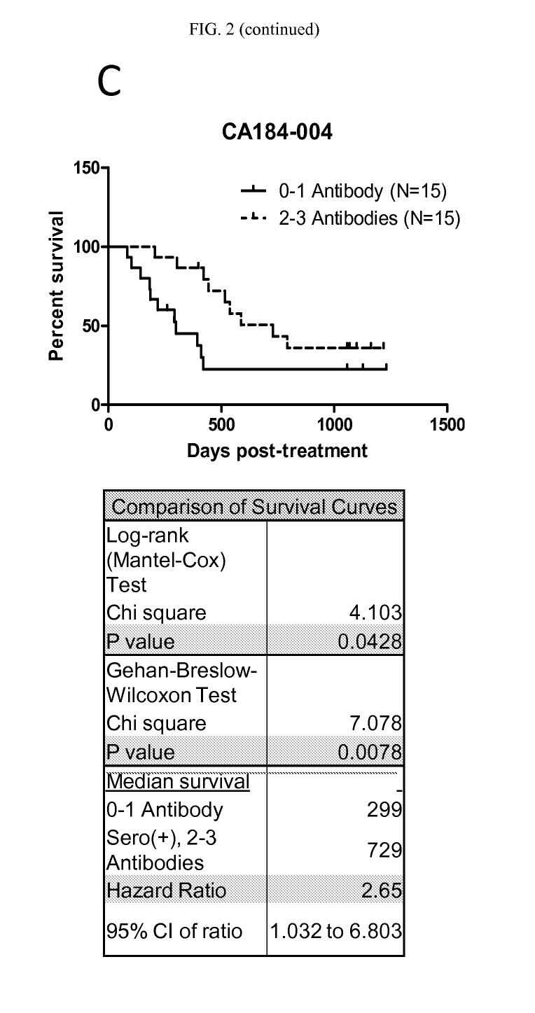

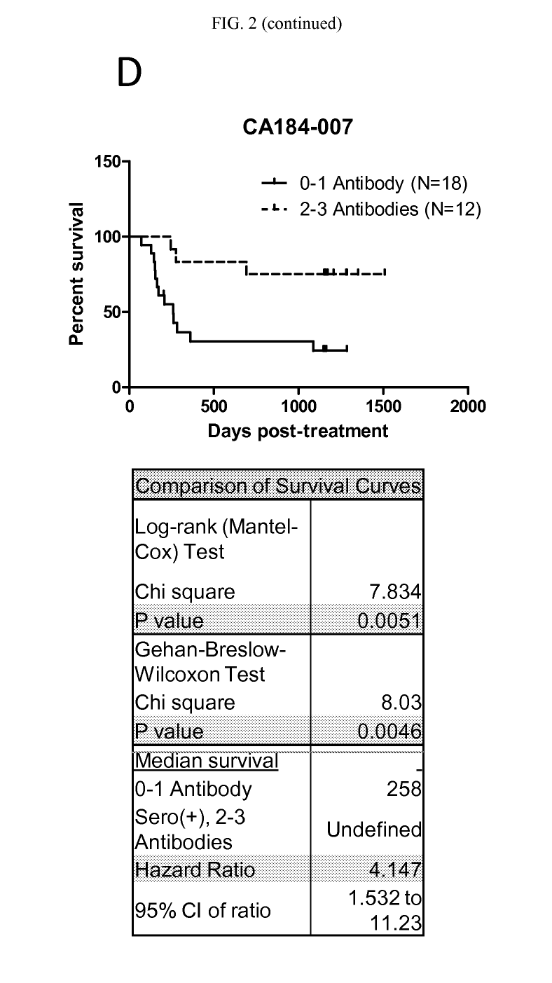

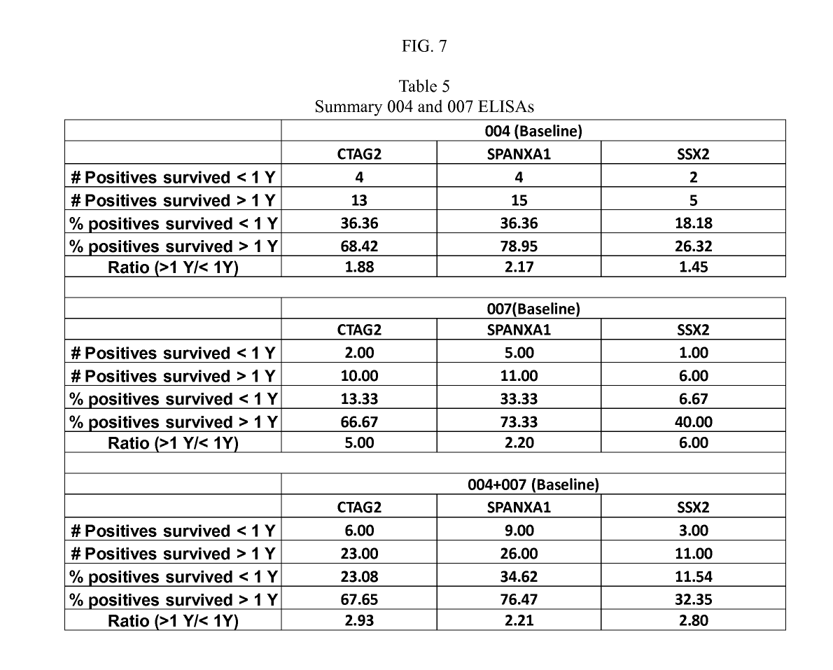

Based on the preliminary array analysis with CA endpoint, a set of 11 antigens was selected for further confirmation by ELISA. Serum samples from two independent clinical trials, CA184-004 and -007 were used as the training and the confirmation datasets, respectively. The criteria for antigen selection were as follows: 1) Amplitude of signal delta from the array experiment at baseline, based on clinical activity; 2) Relative frequency of above-threshold based on clinical activity; 3) Strength of signal on array 4) General interest and previous published data on the specific antigens. This list included TAAs: CABYR, CTAG2, MAGEA1, NLRP4, NYESO1, PBK, SPANXA1, SPANXB1, SSX2, SSX5, TSGA10. The criterion for a positive response was any OD₄₅₀ reading at a dilution of 1:400 of the sera that was above the threshold (defined as average background for each antigen+3×SD). Thirty and 36 months survival data were available for patients treated in CA184-004 and -007, respectively. In sera from CA184-004 high baseline levels of Ab responses toward a number of antigens such as CABYR and NLRP4 (FIG. 5A) were found in most patients independently from their CA status or survival. On the other hand, baseline sero-positivity toward 3 antigens: CTAG2, SPANXA1 and SSX2 were more often found in patients who survived longer than 1 year (FIGS. 2A and 7 (Table 5)).

Findings from CA184-004 were confirmed in an independent data set from phase II clinical trial, CA184-007. Although overall OD₄₅₀ reading intensities were somehow lower in CA184-007 than in CA184-004, similar antibody profiles could be detected and confirmed in this study (FIG. 5B). Hence, patients who had CA or survived longer than 1 year were more often sero-positive toward these antigens, than those with No-CA or shorter survival (FIGS. 2B and 7 (Table 5)).

Kaplan-Meier survival analysis using separate data sets from CA184-004 and -007 showed that patients with Ab responses toward ≧2 of these 3 potential predictive-prognostic antigens: CTAG2, SPANXA1 and SSX2 had greater chance of survival than those with no-response or response to only one antigen. In CA184-004, patients who showed antibody responses toward ≧2 of these antigens had a median survival of 729 days as compared to 299 days for those with Ab responses toward 0-1 of these antigens (p=0.043, HR=0.41) (FIG. 2C). Similarly, in CA184-007, the median survival for patients with baseline Abs toward 0-1 antigen 258 days whereas during the 36 months of the follow up, the median survival was not reached in patients with Abs toward ≧2 antigens (p=0.005) (FIG. 2D).

Treatment with Ipilimumab Results in Significant Increase in Expression of Ig Genes in the Blood

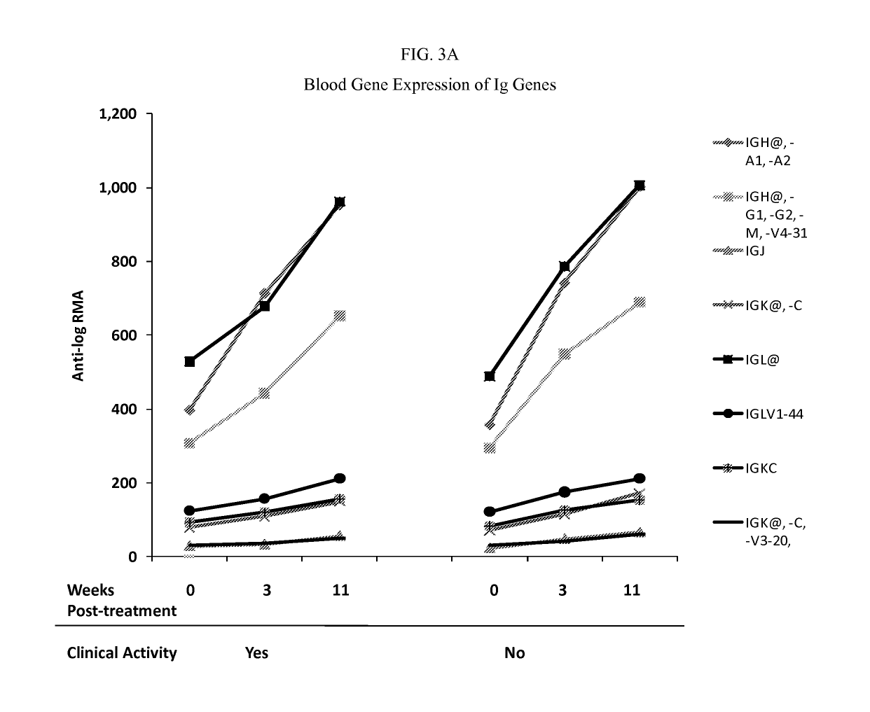

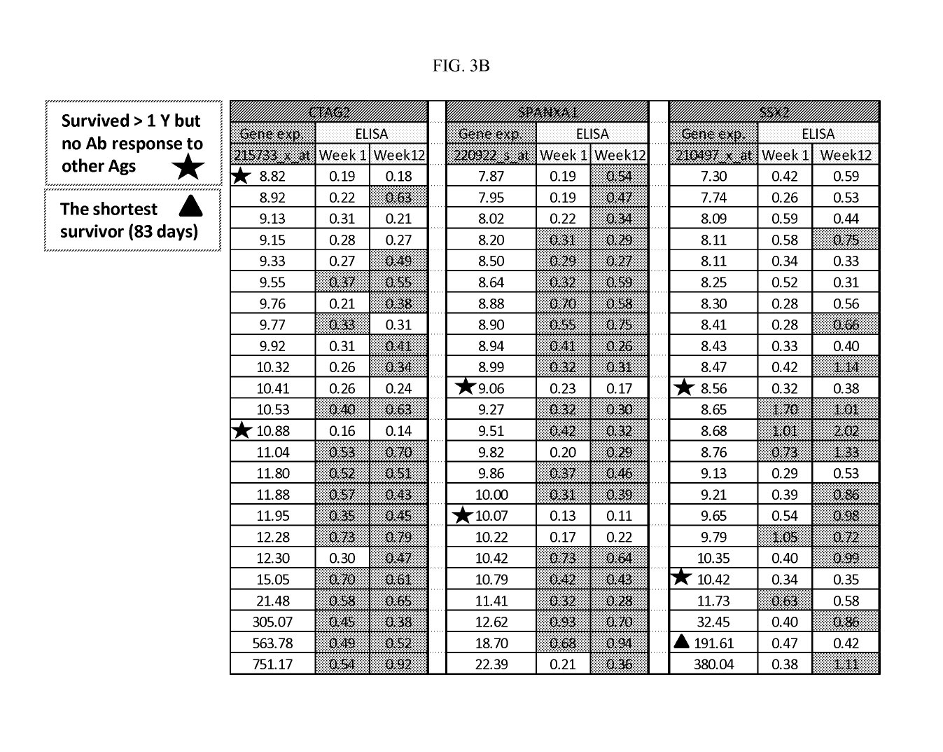

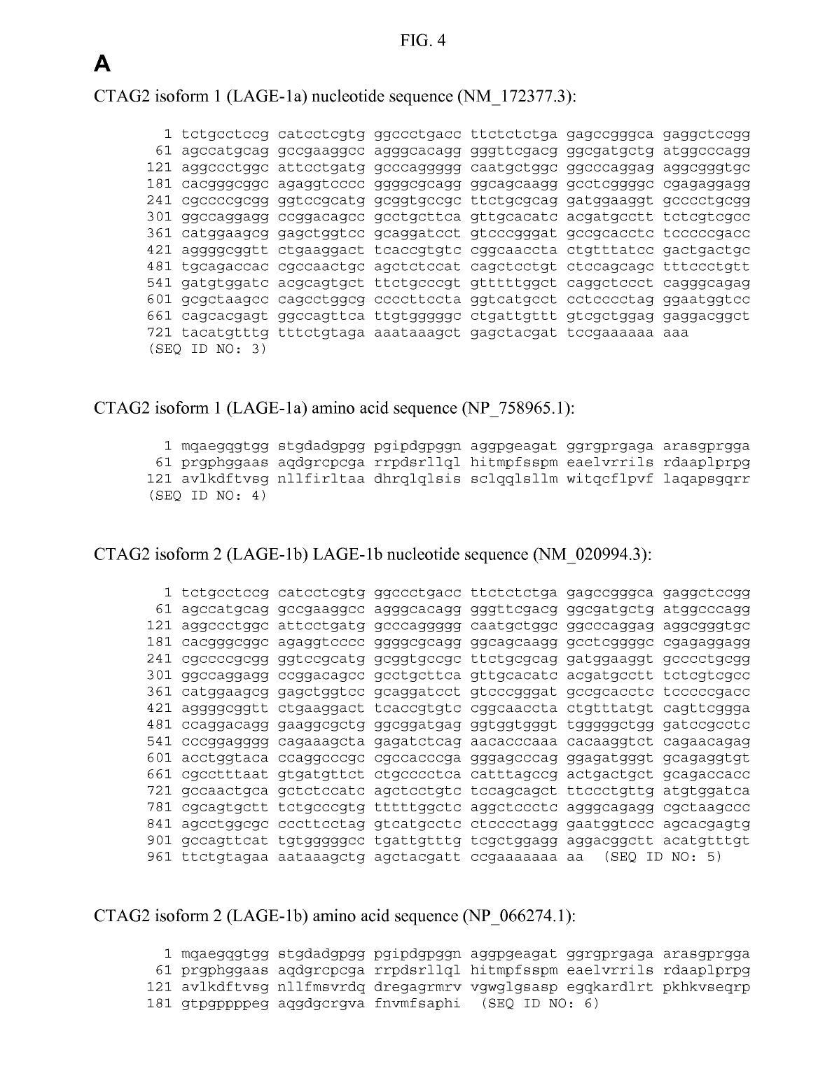

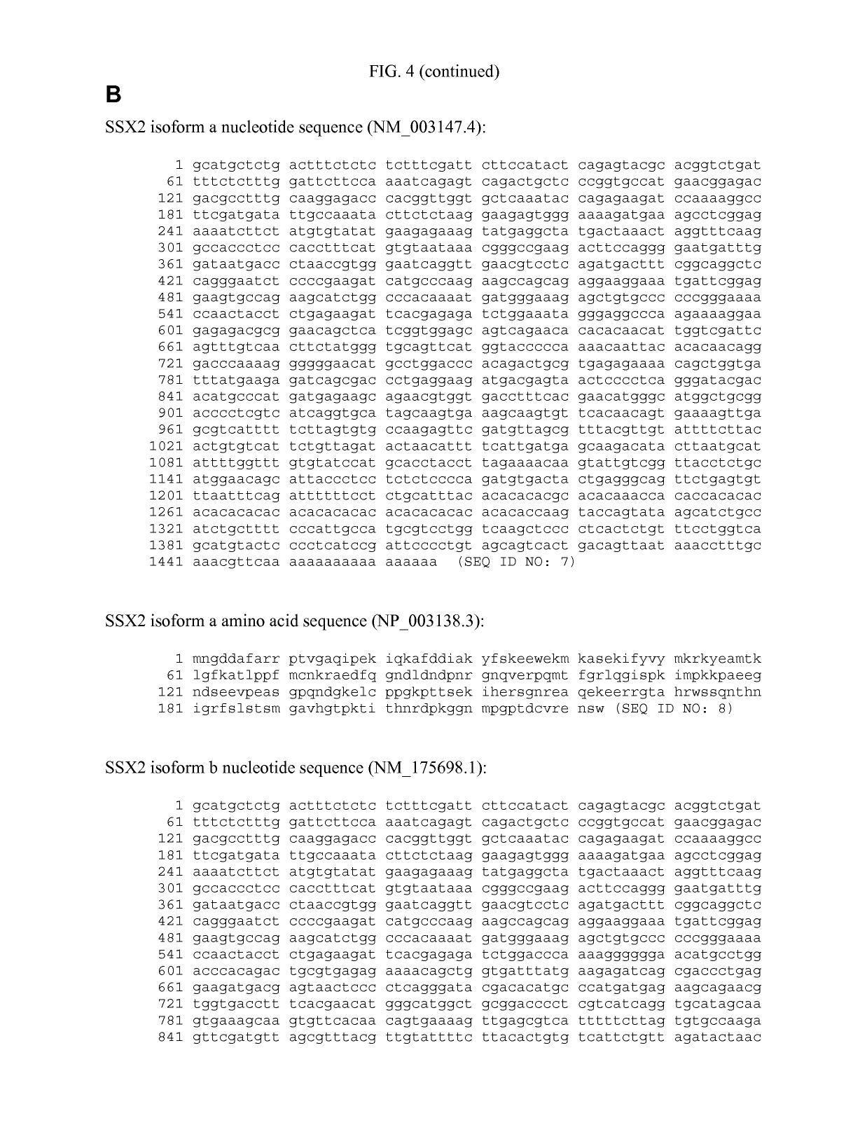

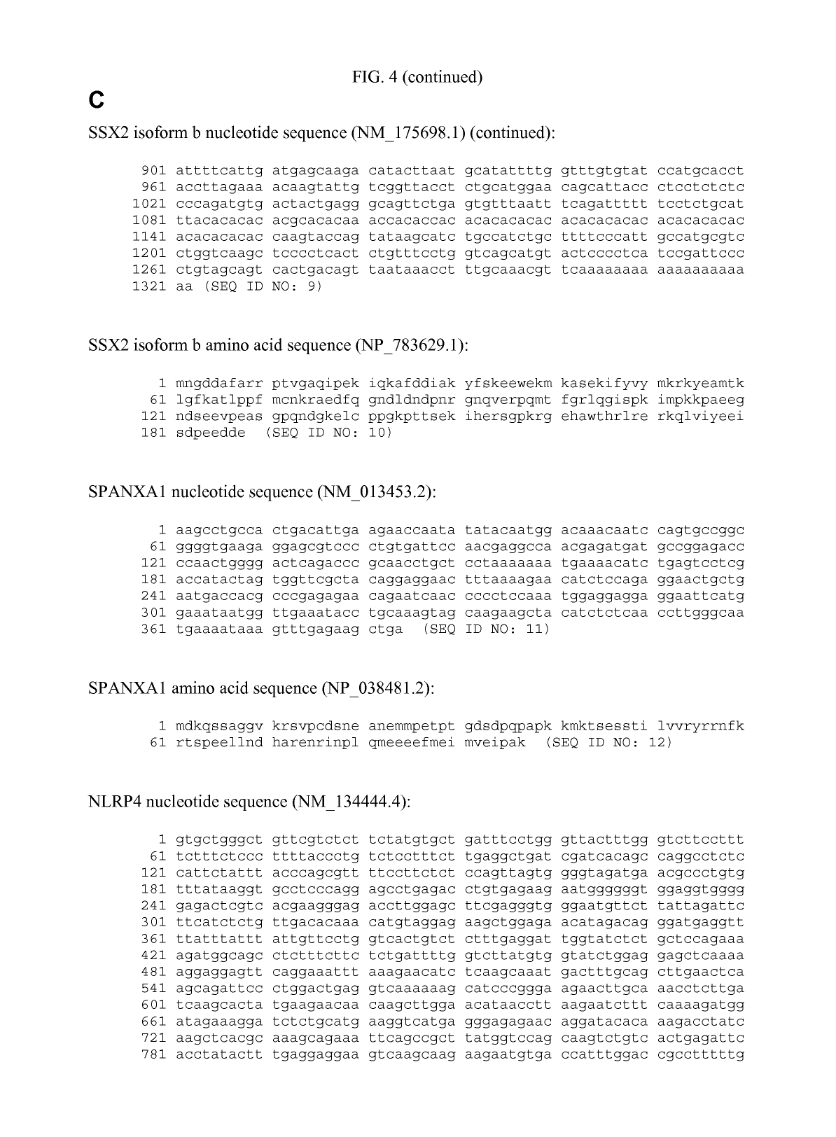

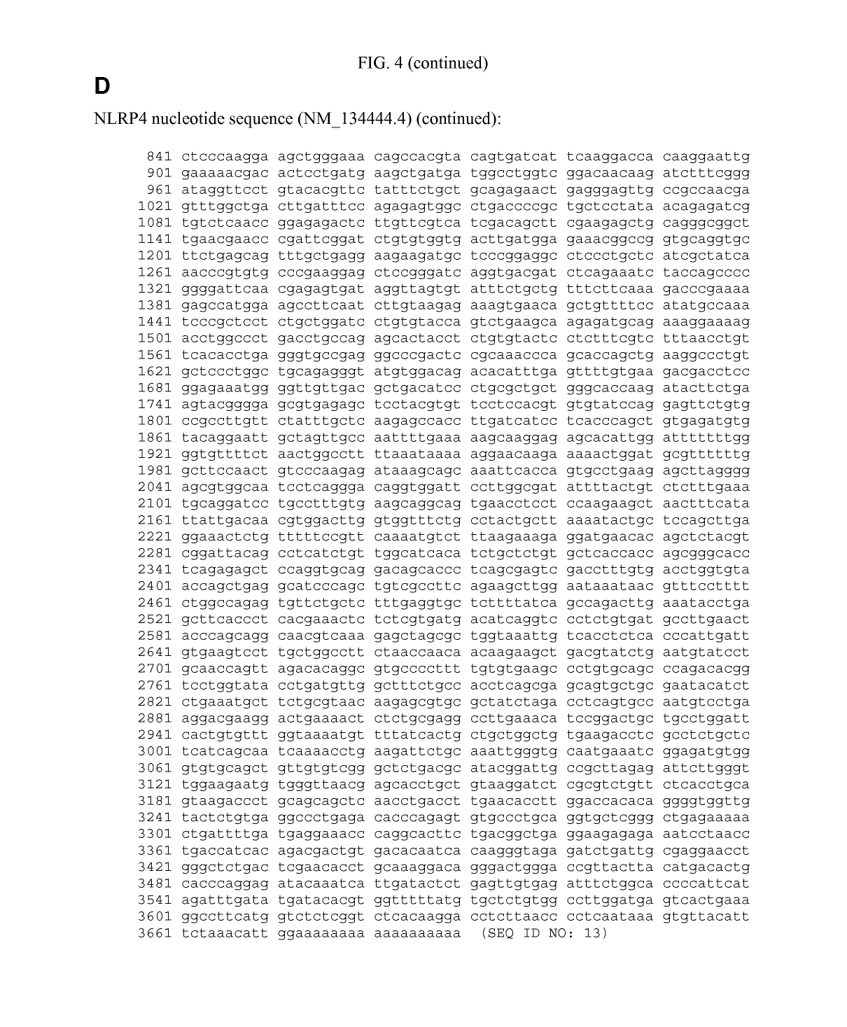

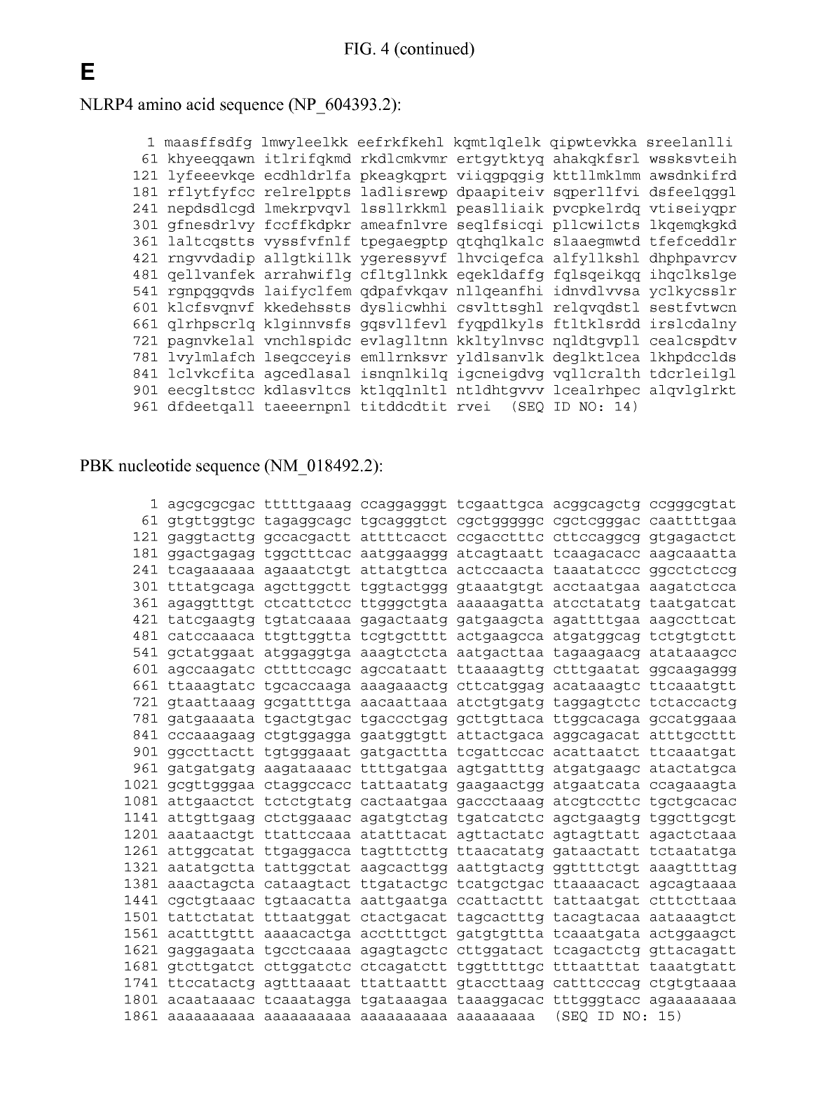

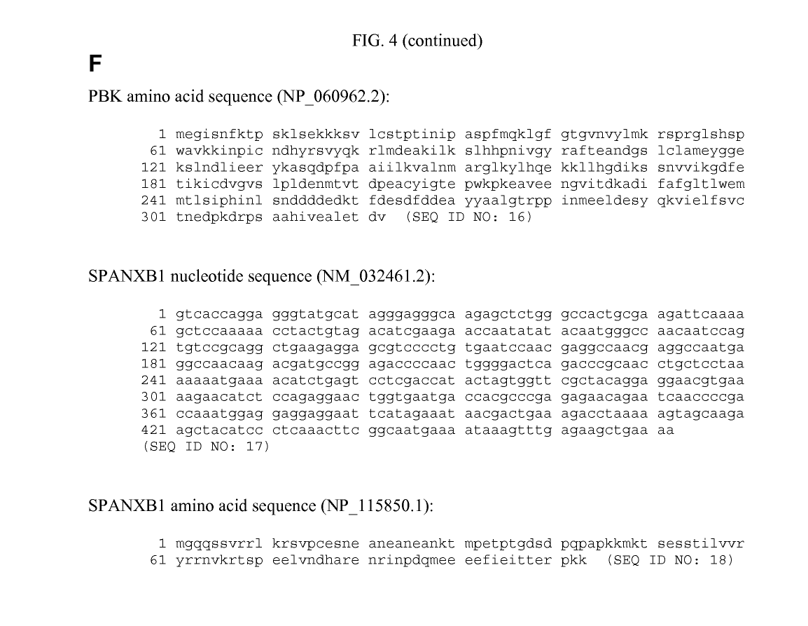

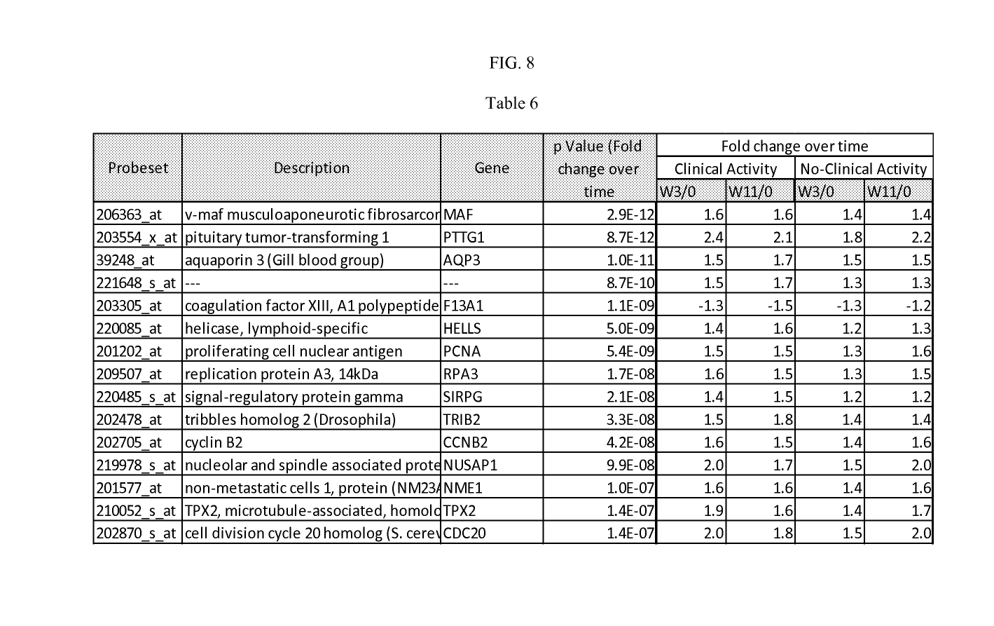

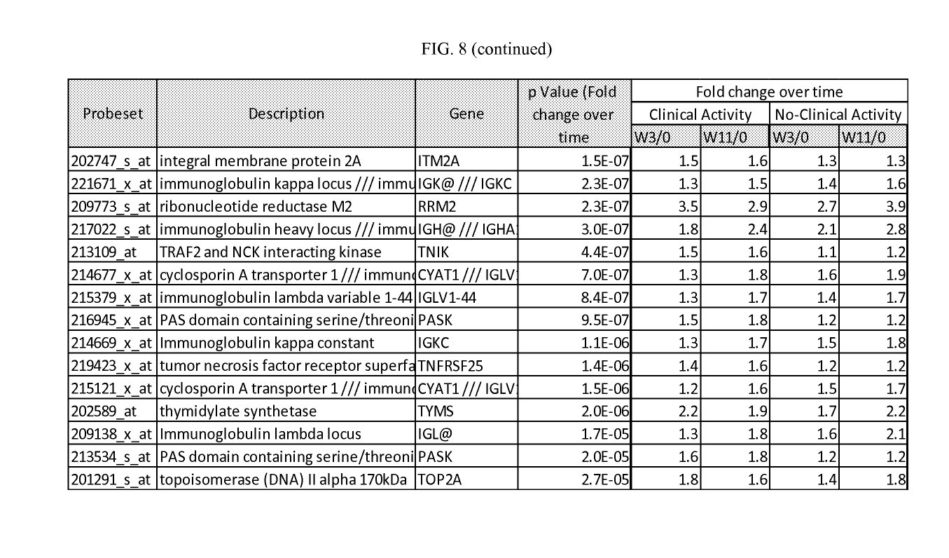

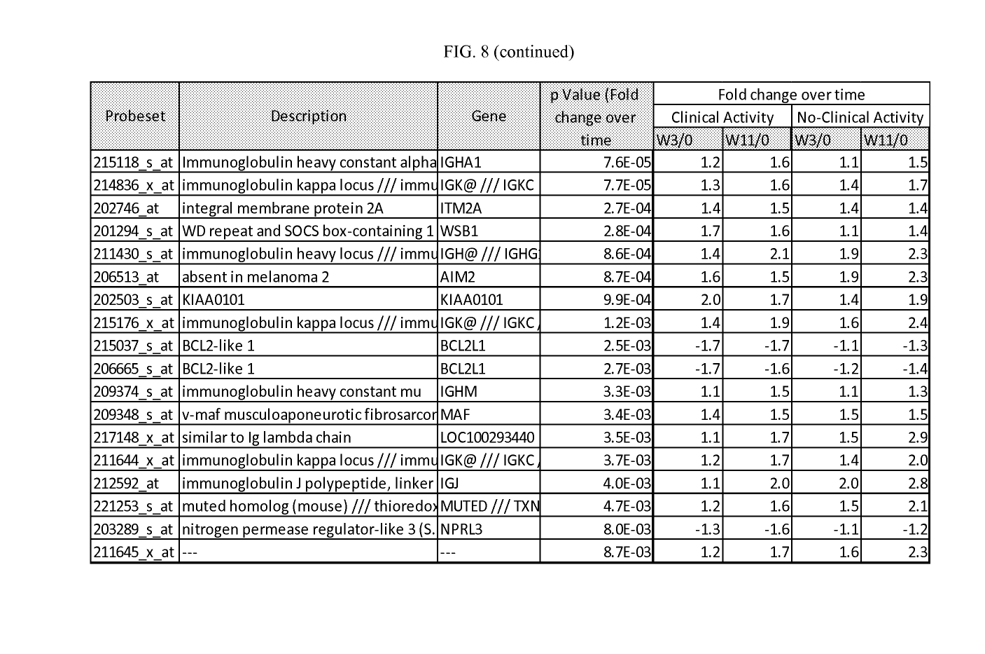

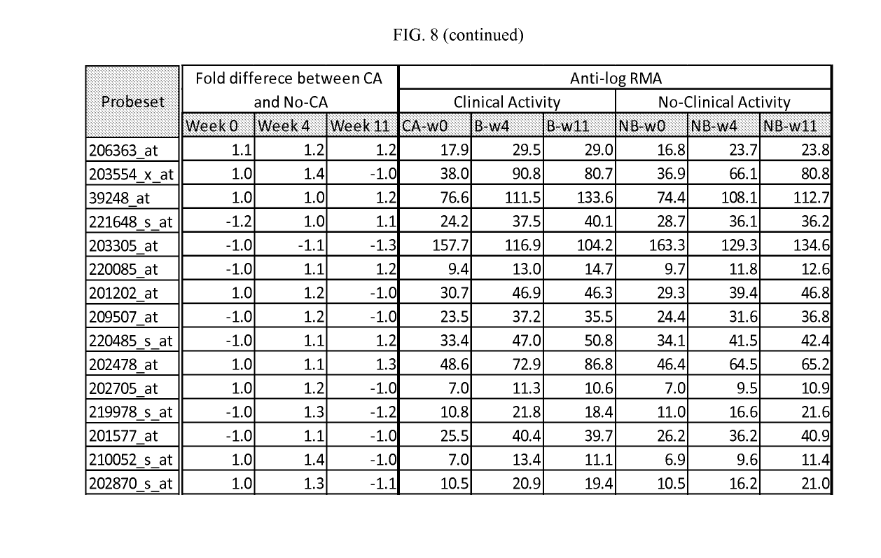

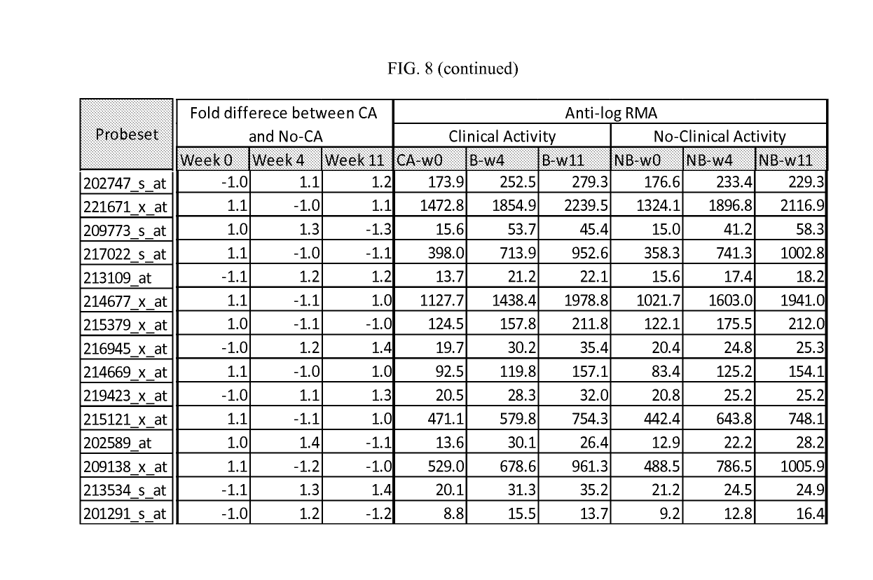

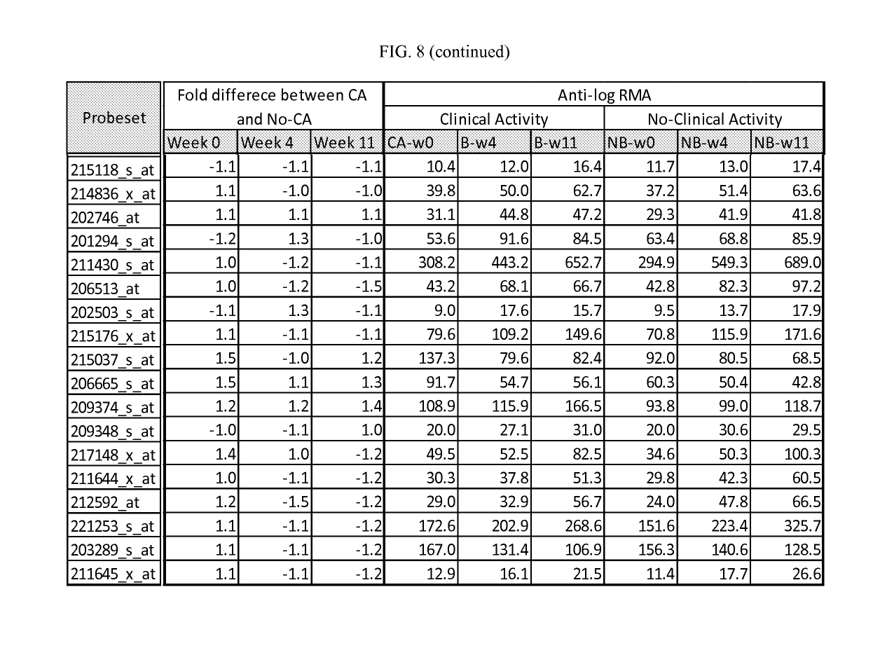

Affymetrix gene expression analysis was performed to assess the pharmacodynamic effects of ipilimumab on the expression profiles of over 22,000 genes in mRNA isolated from peripheral blood. Pooled data from 136 patients treated in two clinical trials CA184-004 and -007 showed that in addition to a number of cell cycle genes such as CCNB2, CDC20 and RRM2, ipilimumab treatment resulted in a significant increase in the expression of several Ig genes from baseline to week 3 and 11 (FIG. 8 (Table 6), FIG. 3A). This amplification of the Ig gene expression occurred in most patients and was not associated with clinical response to ipilimumab treatment. No significant increases were observed in either the MS4A1 (CD20) gene expression suggesting that treatment with ipilimumab did not augment proliferation of B cells. This is in accordance with previous data, showing that treatment with ipilimumab did not affect the frequency of the B cell population in peripheral blood (data not shown).

### Ipilimumab Treatment Increases and Broadens Ab Responses Toward Tumor Antigens

The increase in Ig gene expression in the blood of ipilimumab treated patients detected by Affymetrix suggested that CTLA-4 blockade can somehow affect the humoral responses in melanoma patients. In order to delineate if any of these antibody responses were specific toward MAAs, post-treatment serum samples (Week 11) from patients in CA184-004 and -007 were tested by ELISA using the 11 Ab/TAA panel. Comparison of the post-treatment Ab levels with that of the baseline (FIG. 5) showed that treatment with ipilimumab resulted in an overall increase in both the intensity and the number of antigens toward which the patients were reactive (FIG. 6). Consistent with the gene expression results, the broader antibody responses observed by ELISA occurred in the majority of patients with no apparent associations with their clinical response or survival.

### Association of Tumor Antigen Gene Expression and Serum Antibody Responses

Matching gene expression profiles of metastatic tumor biopsies for 24 out of 30 serum samples tested in CA184-004 were available for these comparison analyses. Although no statistically significant correlations between intratumoral gene expression levels and the intensity or frequency of the antibody responses could be detected in this data set, an apparent pattern was observed. Hence, patients having higher gene expression of the three top candidates (CTAG2, SPANXA1 and SSX2), also showed more frequent baseline antibody responses than those with lower gene expression. Interestingly, a number of patients with detectable antigen expression in their tumors but negative baseline antibody response, sero-converted after treatment with ipilimumab (FIG. 3B). On the other hand, a few patients (marked with star in FIG. 3B), did not show Ab responses to any of the 11 antigens, independently of their survival or antigen expression levels. Interestingly, one patient (marked with triangle) who displayed high SSX2 gene expression in his tumor but did not respond to any of the three potential predictors antigens, was the shortest survivor in the CA184-004.

### Discussion

Identification of predictive biomarkers for clinical response to immunotherapeutics such as ipilimumab has been a major challenge in this field as these agents do not directly target any specific molecules such as BRAF or KRAS on the tumors Immunotherapeutics exert their anti-tumor activity by targeting the regulatory molecules on immune cells, increasing or maintaining the overall activation state of these cells. Most probably, there is no single target on the tumor but a combination of different antigens and immuno-modulatory components in the tumor microenvironment, which will define the responsiveness of the tumor to the immune attack. Our group recently showed that an immune active tumor microenvironment was more favorable for clinical response to the anti-CTLA-4 molecule, ipilimumab (6). Currently, the validation of a tumor immune-signature as a predictive biomarker is a challenging task. Because of the large inter-individual, and -tumor variability, the use of such signatures for patient stratification purposes is still not well understood. In addition, the need for biopsies for tumor immune profiling is another challenge, which makes these biomarkers less attractive. On the other hand, serum collection is a significantly less invasive and more practical procedure giving preferential advantage to these biomarkers.

Blockade of CTLA-4 on T cells by ipilimumab has been shown to increase the activation and proliferation of both CD4⁺ and CD8⁺ T cells, but no major proliferating effect have been observed on CD20⁺ B cells (9). Despite this observation, peripheral blood gene expression profiling shows that increase in the expression of a number of Ig genes is one of the most prominent effects of treatment with ipilimumab. Although anti-tumor antibody responses are considered not to be the main mechanism of tumor lysis by this agent, antibodies might play an indirect role in tumor kill by antibody dependent cytotoxicity (ADCC) and can severe as surrogate biomarkers of the overall immune-activity against tumors. Antibodies against a large number of melanoma antigens have been reported, some of which might serve as predictive or pharmacodynamic (PD) biomarkers for immunotherapeutic agents such as ipilimumab. In a recent report, ipilimumab was shown to increase antibody responses toward 5 melanoma antigens, Melan-A, MAGE-A4, SSX2, and p53 and NY-ESO-1 (9). A broadening of antibody and tumor specific T cell responses to NY-ESO-1 has also been reported and associations with clinical response to ipilimumab were observed (7). However, this antigen is expressed by 30-40% of melanomas, and the reported associations lacked high sensitivity or specificity for predicting clinical response to ipilimumab. The present study was an attempt to broaden the search for predictive biomarkers of survival after treatment with ipilimumab, by looking at serum antibodies toward a large number of TAAs. We first screened a set of serum samples from an ipilimumab monotherapy trial, CA184-004 in which patients were treated with 3 or 10 mg/kg of ipilimumab for 4 doses. Serum samples from baseline and week 11 post-treatment (2 weeks after the 4th dose of ipilimumab) were screened for 37 analytes covering 30 TAAs, which have been reported to be present in melanomas. Whereas serum reactivity toward some antigens such as BRAF was not apparent, others such as TSGA10, SSX5 elicited strongly positive sero-responses in all or the majority of patients. Based on the findings from the array, we chose a smaller panel of 11 antigens for confirmation by ELISA. The criterion for antigen selection was based on the amplitude, strength and relative frequency of the signal and its association with CA. The results from the first set of ELISAs using CA184-004 samples showed differential baseline anti-tumor Ab responses in patients surviving shorter or longer than one year, toward 3 TAAs (i.e., CTAG2, -SSX2 and -SPANX1). To confirm our findings in the first experiment, a second set of serum samples from an independent trial, CA184-007 in which patients were treated with ipilimumab or ipilimumab+prophylactic Budesonide were tested by similar method. The results from the first experiment could be reproduced in the second experiment for the three top candidates. Hence, high baseline sero-reactivity toward ≧2 of these TAAs was found to be potentially predictive of longer survival in ipilimumab treated patients. Interestingly, all three antigens are located on the X chromosome. SSX2, first discovered by the serological analysis of recombinantly expressed clones (SEREX), belongs to the family of highly homologous synovial sarcoma X (SSX) breakpoint proteins. The transcripts of SSX2 gene have been reported in a significant proportion of human melanomas (50%), colon cancers (25%), hepatocarcinomas (30%), and breast carcinoma (20%) but not in normal tissues except for testis. Antibodies against SSX2 have been found approximately 12% of melanoma patients, but not in apparently healthy controls (12). SSX2 has been reported to elicit both humoral and cellular immune responses in cancer patients (9, 13). In the present report, we also found high expression of SSX2 gene in 12.5% (3 out of 24 biopsies) of metastatic melanomas and baseline antibody responses toward SSX2 were prominent in 20% of these patients, with a significant increase after treatment with ipilimumab.

SPANXA1 belongs to the SPNX (sperm protein associated with the nucleus in the X chromosome) gene family, which has been found in several tumors including melanoma, myeloma, glioblastoma, breast carcinoma, ovarian cancer, testicular germ cell tumors, and hematological malignancies. In melanomas, the prevalence of SPANX expression was 80.9%, but with no expression found in normal skin cells (14). The Affymetrix probe set used in our study did not distinguish between several members of this family. However, we were able to detect anti-SPANXA1 antibody responses in 16 baseline and 21 post-treatment patients out of 24 patients for which the SPANX gene expression in tumors was detectable, suggesting the presence of the antigen in most patients.

Finally, CTAG2 is an autoimmunogenic tumor antigen that belongs to the ESO/LAGE family of cancer-testis antigens. This protein is also expressed in a wide array of cancers including melanoma, breast cancer, bladder cancer and prostate cancer and in normal testis tissue. Sero-reactivity toward this family of the TAAs, in particular to NY-ESO-1 has been reported in the past and associations with clinical activity of ipilimumab have also been suggested (7, 8). Nevertheless, in our present study, in contrast to the CTAG2 (another member of the ESO family), we were not able to show any association of antibody responses toward NY-ESO-1 with clinical activity or survival.

In conclusion, our results from this high through put serum analysis suggest that patients with baseline antibody response toward 2 or 3 of these melanoma associated antigens have a more favorable response to ipilimumab and might survive longer after being treated with this immunotherapeutic agent. CTLA-4 blockade by ipilimumab not only increases the intensity of response toward these tumor antigens, it also broadens the anti-TAA responses toward a larger number of melanoma associated antigens. The exact contribution of these antibodies to the anti-tumor effects of ipilimumab is still unclear. However, our results point to the utility of these antibodies as peripheral surrogate biomarkers of an activate immune response against melanomas, which is further intensified by treatment with ipilimumab. It has to be noted that the two trials used in the present study for identification of these potential biomarkers lacked control groups. Therefore, validation of the predictive vs. prognostic value of these biomarkers in a large, controlled trial is still warranted.

### REFERENCES

1. Allison, J. P. et al., “A role for CTLA-4-mediated inhibitory signals in peripheral T cell tolerance?”, Novartis Foundation Symposium, 215:92-98, discussion 98-102, 186-190 (1998).
2. Allison, J. P. et al., “The Yin and Yang of T cell costimulation”, Science, 270:932-933 (1995).
3. Hodi, F. S. et al., “Improved survival with ipilimumab in patients with metastatic melanoma”, The New England Journal of Medicine, 363:711-723 (2010).
4. Robert, C. et al., “Ipilimumab plus dacarbazine for previously untreated metastatic melanoma”, The New England Journal of Medicine, 364:2517-2526 (2011).
5. Berman, D. et al., “Blockade of cytotoxic T-lymphocyte antigen-4 by ipilimumab results in dysregulation of gastrointestinal immunity in patients with advanced melanoma”, Cancer Immun., 10:11 (2010).
6. Ji, R. R. et al., “An immune-active tumor microenvironment favors clinical response to ipilimumab”, Cancer Immunol. Immunother. (2011).
7. Yuan, J. et al., “Integrated NY-ESO-1 antibody and CD8+ T-cell responses correlate with clinical benefit in advanced melanoma patients treated with ipilimumab”, Proceedings of the National Academy of Sciences of the United States of America (2011).
8. Yuan, J. et al., “CTLA-4 blockade increases antigen-specific CD8(+) T cells in prevaccinated patients with melanoma: three cases”, Cancer Immunol. Immunother., 60:1137-1146 (2011).
9. Weber, J. S. et al., “Ipilimumab increases activated T cells and enhances humoral immunity in patients with advanced melanoma”, J. Immunother., 35:89-97 (2012).
10. Hamid, 0. et al., “Association of baseline and on-study tumor biopsy markers with clinical activity in patients (pts) with advanced melanoma treated with ipilimumab”, AJ Clin. Oncol., 27 (Suppl.), Abstract No. 9008 (2009).
11. Weber, J. et al., “A randomized, double-blind, placebo-controlled, phase II study comparing the tolerability and efficacy of ipilimumab administered with or without prophylactic budesonide in patients with unresectable stage III or IV melanoma”, Clin. Cancer Res., 15:5591-5598 (2009).
12. Tureci, O. et al., “The SSX-2 gene, which is involved in the t(X;18) translocation of synovial sarcomas, codes for the human tumor antigen HOM-MEL-40”, Cancer Research, 56:4766-4772 (1996).
13. Bricard, G. et al., “Naturally acquired MAGE-A10- and SSX-2-specific CD8+ T cell responses in patients with hepatocellular carcinoma”, J. Immunol., 174:1709-1716 (2005).
14. Salemi, M. et al., “A high percentage of skin melanoma cells expresses SPANX proteins”, Am. J. Dermatopathol. 31:182-186 (2009).

The entire disclosure of each document cited (including patents, patent applications, journal articles, abstracts, laboratory manuals, books, GENBANK® Accession numbers, SWISS-PROT® Accession numbers, or other disclosures) herein, e.g., in the Background, Detailed Description, Brief Description of the Drawings, and Examples, is hereby incorporated herein by reference in their entirety. Further, the hard copy of the Sequence Listing submitted herewith, in addition to its corresponding Computer Readable Form, are incorporated herein by reference in their entireties.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

1. The method of claim 128, wherein the cancer is melanoma, and the method comprises
identifying a subject having melanoma and having a level of antibodies to each of at least two tumor associated antigens (TAAs) that is higher than a predetermined antibody value for each of the two TAAs; and
administering to the subject a therapeutically effective amount of a therapeutic agent for treating melanoma.

2. The method of claim 1, wherein the at least two TAAs are selected from the group consisting of CTAG2, SSX2, SPANXA1, NLRP4, PBK and SPANXB1.

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. The method of claim 128, for treating a subject having cancer with ipilimumab, comprising
identifying a subject having cancer and having a level of antibodies to each of at least two TAAs selected from the group consisting of CTAG2, SSX2, SPANXA1, NLRP4, PBK and SPANXB1 that is higher than a predetermined antibody value for each of the two TAAs; and
administering to the subject a therapeutically effective amount of ipilimumab.

16. (canceled)

17. The method of claim 16, wherein the at least two TAAs are selected from the group consisting of CTAG2, SSX2 and SPANXA1.

18. (canceled)

19. (canceled)

20. (canceled)

21. The method of claim 128, wherein the cancer is melanoma, and the method comprises
determining the level of antibodies to each of at least two TAAs in a subject having melanoma; and
administering to the subject a therapeutically effective dose of a therapeutic agent for treating melanoma if the level of antibodies to each of at least two TAAs in the subject is higher than a predetermined antibody value for each of the two TAAs.

22. The method of claim 21, wherein the at least two TAAs are selected from the group consisting of CTAG2, SSX2, SPANXA1, NLRP4, PBK and SPANXB1.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. The method of claim 128, for treating a subject having cancer with ipilimumab, comprising
determining the level of antibodies to each of at least two TAAs selected from the group consisting of CTAG2, SSX2, SPANXA1, NLRP4, PBK and SPANXB1 in a subject having cancer; and
administering to the subject a therapeutically effective dose of ipilimumab if the level of antibodies to each of at least two TAAs in the subject is higher than a predetermined antibody value for each of the two TAAs.

36. (canceled)

37. The method of claim 36, wherein the at least two TAAs are selected from the group consisting of CTAG2, SSX2 and SPANXA1.

38. (canceled)

39. (canceled)

40. (canceled)

41. The method of claim 129, wherein the method is for determining whether a subject having melanoma is likely to respond to a therapeutic agent for treating melanoma, comprising
determining the level of antibodies to each of at least two TAAs in a subject having melanoma, wherein
a higher level of antibodies to each of at least two TAAs in the subject having melanoma relative to a predetermined antibody value for each TAA indicates that the subject is likely to respond to a therapeutic agent for treating melanoma; and
the absence of a higher level of antibodies to each of at least two TAAs in the subject having melanoma relative to a predetermined antibody value for each TAA indicates that the subject is not likely to respond to a therapeutic agent for treating melanoma.

42. The method of claim 41, wherein the at least two TAAs are selected from the group consisting of CTAG2, SSX2, SPANXA1, NLRP4, PBK and SPANXB1.

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. The method of claim 129, wherein the method is for determining whether a subject having cancer is likely to respond to treatment with ipilimumab, comprising
determining the level of antibodies to each of at least two TAAs in a subject having cancer, wherein
a higher level of antibodies to each of at least two TAAs selected from the group consisting of CTAG2, SSX2, SPANXA1, NLRP4, PBK and SPANXB1 in the subject having cancer relative to a predetermined antibody value for each TAA indicates that the subject is likely to respond to treatment with ipilimumab; and
the absence of a higher level of antibodies to each of at least two TAAs selected from the group consisting of CTAG2, SSX2, SPANXA1, NLRP4, PBK and SPANXB 1 in the subject having cancer relative to a predetermined antibody value for each TAA indicates that the subject is not likely to respond to treatment with ipilimumab.

56. (canceled)

57. The method of claim 56, wherein the at least two TAAs are selected from the group consisting of CTAG2, SSX2 and SPANXA1.

58. (canceled)

59. (canceled)

60. (canceled)

61. The method of claim 129, wherein the method is for determining whether to treat a subject having melanoma with a therapeutic agent for melanoma, comprising
determining the level of antibodies to each of at least two TAAs in a subject having melanoma, wherein
a higher level of antibodies to each of at least two TAAs relative to a predetermined antibody value for each of the TAAs indicates that the subject should be treated with a therapeutic agent for treating melanoma; and
the absence of a higher level of antibodies to each of at least two TAAs relative to a predetermined antibody value for each of the TAAs indicates that the subject should not be treated with a therapeutic agent for treating melanoma.

62. The method of claim 61, wherein the at least two TAAs are selected from the group consisting of CTAG2, SSX2, SPANXA1, NLRP4, PBK and SPANXB1.

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. The method of claim 129, wherein the method is for determining whether to treat a subject having cancer with ipilimumab, comprising
determining the level of antibodies to each of at least two TAAs selected from the group consisting of CTAG2, SSX2, SPANXA1, NLRP4, PBK and SPANXB1 in a subject having cancer, wherein
a higher level of antibodies to each of two TAAs relative to a predetermined antibody value for each of the TAAs indicates that the subject should be treated with ipilimumab; and
the absence of a higher level of antibodies to each of two TAAs relative to a predetermined antibody value for each of the TAAs indicates that the subject should not be treated with ipilimumab.

76. (canceled)

77. The method of claim 76, wherein the at least two TAAs are selected from the group consisting of CTAG2, SSX2 and SPANXA1.

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. A method for determining the level of antibodies to each of at least two TAAs selected from the group consisting of CTAG2, SSX2, SPANXA1, NLRP4, PBK and SPANXB1 in a serum sample of a human subject, comprising
providing a serum sample from a human subject; and
measuring the level of antibodies to each of at least two TAAs selected from the group consisting of CTAG2, SSX2, SPANXA1, NLRP4, PBK and SPANXB1.

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. A method for treating a subject having cancer, comprising
identifying a subject having cancer and having a level of antibodies to each of at least two tumor associated antigens (TAAs) that is higher than a predetermined antibody value for each of the two TAAs; and
administering to the subject a therapeutically effective amount of a therapeutic agent for treating cancer.

129. A method for determining whether a subject having cancer is likely to respond to a therapeutic agent for treating cancer and/or whether to treat the subject with a therapeutic agent for treating cancer, comprising
determining the level of antibodies to each of at least two TAAs in a subject having cancer, wherein
a higher level of antibodies to each of at least two TAAs in the subject having cancer relative to a predetermined antibody value for each TAA indicates that the subject is likely to respond to a therapeutic agent for treating cancer and/or that the subject should be treated with a therapeutic agent for treating cancer; and
the absence of a higher level of antibodies to each of at least two TAAs in the subject having cancer relative to a predetermined antibody value for each TAA indicates that the subject is not likely to respond to a therapeutic agent for treating cancer and/or should not be treated with a therapeutic agent for treating cancer.


SEQUENCE LISTING (18 sequences)

SEQ ID NO: 1 - protein, 108 aa, Artificial Sequence
  Synthesized polypeptide
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 2 - protein, 118 aa, Artificial Sequence
  Synthesized polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 3 - DNA, 773 bp, Homo sapiens
tctgcctccgcatcctcgtgggccctgaccttctctctgagagccgggcagaggctccggagccatgcaggccgaaggccagggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgagaggaggcgccccgcggggtccgcatggcggtgccgcttctgcgcaggatggaaggtgcccctgcggggccaggaggccggacagccgcctgcttcagttgcacatcacgatgcctttctcgtcgcccatggaagcggagctggtccgcaggatcctgtcccgggatgccgcacctctcccccgaccaggggcggttctgaaggacttcaccgtgtccggcaacctactgtttatccgactgactgctgcagaccaccgccaactgcagctctccatcagctcctgtctccagcagctttccctgttgatgtggatcacgcagtgctttctgcccgtgtttttggctcaggctccctcagggcagaggcgctaagcccagcctggcgccccttcctaggtcatgcctcctcccctagggaatggtcccagcacgagtggccagttcattgtgggggcctgattgtttgtcgctggaggaggacggcttacatgtttgtttctgtagaaaataaagctgagctacgattccgaaaaaaaaa

SEQ ID NO: 4 - protein, 180 aa, Homo sapiens
MQAEGQGTGGSTGDADGPGGPGIPDGPGGNAGGPGEAGATGGRGPRGAGAARASGPRGGAPRGPHGGAASAQDGRCPCGARRPDSRLLQLHITMPFSSPMEAELVRRILSRDAAPLPRPGAVLKDFTVSGNLLFIRLTAADHRQLQLSISSCLQQLSLLMWITQCFLPVFLAQAPSGQRR

SEQ ID NO: 5 - DNA, 1002 bp, Homo sapiens
tctgcctccgcatcctcgtgggccctgaccttctctctgagagccgggcagaggctccggagccatgcaggccgaaggccagggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgagaggaggcgccccgcggggtccgcatggcggtgccgcttctgcgcaggatggaaggtgcccctgcggggccaggaggccggacagccgcctgcttcagttgcacatcacgatgcctttctcgtcgcccatggaagcggagctggtccgcaggatcctgtcccgggatgccgcacctctcccccgaccaggggcggttctgaaggacttcaccgtgtccggcaacctactgtttatgtcagttcgggaccaggacagggaaggcgctgggcggatgagggtggtgggttgggggctgggatccgcctccccggaggggcagaaagctagagatctcagaacacccaaacacaaggtctcagaacagagacctggtacaccaggcccgccgccacccgagggagcccagggagatgggtgcagaggtgtcgcctttaatgtgatgttctctgcccctcacatttagccgactgactgctgcagaccaccgccaactgcagctctccatcagctcctgtctccagcagctttccctgttgatgtggatcacgcagtgctttctgcccgtgtttttggctcaggctccctcagggcagaggcgctaagcccagcctggcgccccttcctaggtcatgcctcctcccctagggaatggtcccagcacgagtggccagttcattgtgggggcctgattgtttgtcgctggaggaggacggcttacatgtttgtttctgtagaaaataaagctgagctacgattccgaaaaaaaaa

SEQ ID NO: 6 - protein, 210 aa, Homo sapiens
MQAEGQGTGGSTGDADGPGGPGIPDGPGGNAGGPGEAGATGGRGPRGAGAARASGPRGGAPRGPHGGAASAQDGRCPCGARRPDSRLLQLHITMPFSSPMEAELVRRILSRDAAPLPRPGAVLKDFTVSGNLLFMSVRDQDREGAGRMRVVGWGLGSASPEGQKARDLRTPKHKVSEQRPGTPGPPPPEGAQGDGCRGVAFNVMFSAPHI

SEQ ID NO: 7 - DNA, 1466 bp, Homo sapiens
gcatgctctgactttctctctctttcgattcttccatactcagagtacgcacggtctgattttctctttggattcttccaaaatcagagtcagactgctcccggtgccatgaacggagacgacgcctttgcaaggagacccacggttggtgctcaaataccagagaagatccaaaaggccttcgatgatattgccaaatacttctctaaggaagagtgggaaaagatgaaagcctcggagaaaatcttctatgtgtatatgaagagaaagtatgaggctatgactaaactaggtttcaaggccaccctcccacctttcatgtgtaataaacgggccgaagacttccaggggaatgatttggataatgaccctaaccgtgggaatcaggttgaacgtcctcagatgactttcggcaggctccagggaatctccccgaagatcatgcccaagaagccagcagaggaaggaaatgattcggaggaagtgccagaagcatctggcccacaaaatgatgggaaagagctgtgccccccgggaaaaccaactacctctgagaagattcacgagagatctggaaatagggaggcccaagaaaaggaagagagacgcggaacagctcatcggtggagcagtcagaacacacacaacattggtcgattcagtttgtcaacttctatgggtgcagttcatggtacccccaaaacaattacacacaacagggacccaaaaggggggaacatgcctggacccacagactgcgtgagagaaaacagctggtgatttatgaagagatcagcgaccctgaggaagatgacgagtaactcccctcagggatacgacacatgcccatgatgagaagcagaacgtggtgacctttcacgaacatgggcatggctgcggacccctcgtcatcaggtgcatagcaagtgaaagcaagtgttcacaacagtgaaaagttgagcgtcatttttcttagtgtgccaagagttcgatgttagcgtttacgttgtattttcttacactgtgtcattctgttagatactaacattttcattgatgagcaagacatacttaatgcatattttggtttgtgtatccatgcacctaccttagaaaacaagtattgtcggttacctctgcatggaacagcattaccctcctctctccccagatgtgactactgagggcagttctgagtgtttaatttcagattttttcctctgcatttacacacacacgcacacaaaccacaccacacacacacacacacacacacacacacacacacacacacaccaagtaccagtataagcatctgccatctgcttttcccattgccatgcgtcctggtcaagctcccctcactctgtttcctggtcagcatgtactcccctcatccgattcccctgtagcagtcactgacagttaataaacctttgcaaacgttcaaaaaaaaaaaaaaaaaa

SEQ ID NO: 8 - protein, 223 aa, Homo sapiens
MNGDDAFARRPTVGAQIPEKIQKAFDDIAKYFSKEEWEKMKASEKIFYVYMKRKYEAMTKLGFKATLPPFMCNKRAEDFQGNDLDNDPNRGNQVERPQMTFGRLQGISPKIMPKKPAEEGNDSEEVPEASGPQNDGKELCPPGKPTTSEKIHERSGNREAQEKEERRGTAHRWSSQNTHNIGRFSLSTSMGAVHGTPKTITHNRDPKGGNMPGPTDCVRENSW

SEQ ID NO: 9 - DNA, 1322 bp, Homo sapiens
gcatgctctgactttctctctctttcgattcttccatactcagagtacgcacggtctgattttctctttggattcttccaaaatcagagtcagactgctcccggtgccatgaacggagacgacgcctttgcaaggagacccacggttggtgctcaaataccagagaagatccaaaaggccttcgatgatattgccaaatacttctctaaggaagagtgggaaaagatgaaagcctcggagaaaatcttctatgtgtatatgaagagaaagtatgaggctatgactaaactaggtttcaaggccaccctcccacctttcatgtgtaataaacgggccgaagacttccaggggaatgatttggataatgaccctaaccgtgggaatcaggttgaacgtcctcagatgactttcggcaggctccagggaatctccccgaagatcatgcccaagaagccagcagaggaaggaaatgattcggaggaagtgccagaagcatctggcccacaaaatgatgggaaagagctgtgccccccgggaaaaccaactacctctgagaagattcacgagagatctggacccaaaaggggggaacatgcctggacccacagactgcgtgagagaaaacagctggtgatttatgaagagatcagcgaccctgaggaagatgacgagtaactcccctcagggatacgacacatgcccatgatgagaagcagaacgtggtgacctttcacgaacatgggcatggctgcggacccctcgtcatcaggtgcatagcaagtgaaagcaagtgttcacaacagtgaaaagttgagcgtcatttttcttagtgtgccaagagttcgatgttagcgtttacgttgtattttcttacactgtgtcattctgttagatactaacattttcattgatgagcaagacatacttaatgcatattttggtttgtgtatccatgcacctaccttagaaaacaagtattgtcggttacctctgcatggaacagcattaccctcctctctccccagatgtgactactgagggcagttctgagtgtttaatttcagattttttcctctgcatttacacacacacgcacacaaaccacaccacacacacacacacacacacacacacacacacacacacacaccaagtaccagtataagcatctgccatctgcttttcccattgccatgcgtcctggtcaagctcccctcactctgtttcctggtcagcatgtactcccctcatccgattcccctgtagcagtcactgacagttaataaacctttgcaaacgttcaaaaaaaaaaaaaaaaaaaa

SEQ ID NO: 10 - protein, 188 aa, Homo sapiens
MNGDDAFARRPTVGAQIPEKIQKAFDDIAKYFSKEEWEKMKASEKIFYVYMKRKYEAMTKLGFKATLPPFMCNKRAEDFQGNDLDNDPNRGNQVERPQMTFGRLQGISPKIMPKKPAEEGNDSEEVPEASGPQNDGKELCPPGKPTTSEKIHERSGPKRGEHAWTHRLRERKQLVIYEEISDPEEDDE

SEQ ID NO: 11 - DNA, 384 bp, Homo sapiens
aagcctgccactgacattgaagaaccaatatatacaatggacaaacaatccagtgccggcggggtgaagaggagcgtcccctgtgattccaacgaggccaacgagatgatgccggagaccccaactggggactcagacccgcaacctgctcctaaaaaaatgaaaacatctgagtcctcgaccatactagtggttcgctacaggaggaactttaaaagaacatctccagaggaactgctgaatgaccacgcccgagagaacagaatcaaccccctccaaatggaggaggaggaattcatggaaataatggttgaaatacctgcaaagtagcaagaagctacatctctcaaccttgggcaatgaaaataaagtttgagaagctga

SEQ ID NO: 12 - protein, 97 aa, Homo sapiens
MDKQSSAGGVKRSVPCDSNEANEMMPETPTGDSDPQPAPKKMKTSESSTILVVRYRRNFKRTSPEELLNDHARENRINPLQMEEEEFMEIMVEIPAK

SEQ ID NO: 13 - DNA, 3690 bp, Homo sapiens
gtgctgggctgttcgtctcttctatgtgctgatttcctgggttactttgggtcttccttttctttctcccttttaccctgtctcctttcttgaggctgatcgatcacagccaggcctctccattctatttacccagcgttttccttctctccagttagtggggtagatgaacgccctgtgtttataaggtgcctcccaggagcctgagacctgtgagaagaatggggggtggaggtgggggagactcgtcacgaagggagaccttggagcttcgagggtgggaatgttcttattagattcttcatctctgttgacacaaacatgtaggagaagctggagaacatagacagggatgaggttttatttatttattgttcctggtcactgtctctttgaggattggtatctctgctccagaaaagatggcagcctctttcttctctgattttggtcttatgtggtatctggaggagctcaaaaaggaggagttcaggaaatttaaagaacatctcaagcaaatgactttgcagcttgaactcaagcagattccctggactgaggtcaaaaaagcatcccgggaagaacttgcaaacctcttgatcaagcactatgaagaacaacaagcttggaacataaccttaagaatctttcaaaagatggatagaaaggatctctgcatgaaggtcatgagggagagaacaggatacacaaagacctatcaagctcacgcaaagcagaaattcagccgcttatggtccagcaagtctgtcactgagattcacctatactttgaggaggaagtcaagcaagaagaatgtgaccatttggaccgcctttttgctcccaaggaagctgggaaacagccacgtacagtgatcattcaaggaccacaaggaattggaaaaacgacactcctgatgaagctgatgatggcctggtcggacaacaagatctttcgggataggttcctgtacacgttctatttctgctgcagagaactgagggagttgccgccaacgagtttggctgacttgatttccagagagtggcctgaccccgctgctcctataacagagatcgtgtctcaaccggagagactcttgttcgtcatcgacagcttcgaagagctgcagggcggcttgaacgaacccgattcggatctgtgtggtgacttgatggagaaacggccggtgcaggtgcttctgagcagtttgctgaggaagaagatgctcccggaggcctccctgctcatcgctatcaaacccgtgtgcccgaaggagctccgggatcaggtgacgatctcagaaatctaccagccccggggattcaacgagagtgataggttagtgtatttctgctgtttcttcaaagacccgaaaagagccatggaagccttcaatcttgtaagagaaagtgaacagctgttttccatatgccaaatcccgctcctctgctggatcctgtgtaccagtctgaagcaagagatgcagaaaggaaaagacctggccctgacctgccagagcactacctctgtgtactcctctttcgtctttaacctgttcacacctgagggtgccgagggcccgactccgcaaacccagcaccagctgaaggccctgtgctccctggctgcagagggtatgtggacagacacatttgagttttgtgaagacgacctccggagaaatggggttgttgacgctgacatccctgcgctgctgggcaccaagatacttctgaagtacggggagcgtgagagctcctacgtgttcctccacgtgtgtatccaggagttctgtgccgccttgttctatttgctcaagagccaccttgatcatcctcacccagctgtgagatgtgtacaggaattgctagttgccaattttgaaaaagcaaggagagcacattggatttttttggggtgttttctaactggccttttaaataaaaaggaacaagaaaaactggatgcgttttttggcttccaactgtcccaagagataaagcagcaaattcaccagtgcctgaagagcttaggggagcgtggcaatcctcagggacaggtggattccttggcgatattttactgtctctttgaaatgcaggatcctgcctttgtgaagcaggcagtgaacctcctccaagaagctaactttcatattattgacaacgtggacttggtggtttctgcctactgcttaaaatactgctccagcttgaggaaactctgtttttccgttcaaaatgtctttaagaaagaggatgaacacagctctacgtcggattacagcctcatctgttggcatcacatctgctctgtgctcaccaccagcgggcacctcagagagctccaggtgcaggacagcaccctcagcgagtcgacctttgtgacctggtgtaaccagctgaggcatcccagctgtcgccttcagaagcttggaataaataacgtttccttttctggccagagtgttctgctctttgaggtgctcttttatcagccagacttgaaatacctgagcttcaccctcacgaaactctctcgtgatgacatcaggtccctctgtgatgccttgaactacccagcaggcaacgtcaaagagctagcgctggtaaattgtcacctctcacccattgattgtgaagtccttgctggccttctaaccaacaacaagaagctgacgtatctgaatgtatcctgcaaccagttagacacaggcgtgccccttttgtgtgaagccctgtgcagcccagacacggtcctggtatacctgatgttggctttctgccacctcagcgagcagtgctgcgaatacatctctgaaatgcttctgcgtaacaagagcgtgcgctatctagacctcagtgccaatgtcctgaaggacgaaggactgaaaactctctgcgaggccttgaaacatccggactgctgcctggattcactgtgtttggtaaaatgttttatcactgctgctggctgtgaagacctcgcctctgctctcatcagcaatcaaaacctgaagattctgcaaattgggtgcaatgaaatcggagatgtgggtgtgcagctgttgtgtcgggctctgacgcatacggattgccgcttagagattcttgggttggaagaatgtgggttaacgagcacctgctgtaaggatctcgcgtctgttctcacctgcagtaagaccctgcagcagctcaacctgaccttgaacaccttggaccacacaggggtggttgtactctgtgaggccctgagacacccagagtgtgccctgcaggtgctcgggctgagaaaaactgattttgatgaggaaacccaggcacttctgacggctgaggaagagagaaatcctaacctgaccatcacagacgactgtgacacaatcacaagggtagagatctgattgcgaggaacctgggctctgactcgaacacctgcaaaggacagggactgggaccgttacttacatgacactgcacccaggagatacaaatcattgatactctgagttgtgagatttctggcaccccattcatagatttgatatgatacacgtggtttttatgtgctctgtggccttggatgagtcactgaaaggccttcatggtctctcggtctcacaaggacctcttaacccctcaataaagtgttacatttctaaacattggaaaaaaaaaaaaaaaaaa

SEQ ID NO: 14 - protein, 994 aa, Homo sapiens
MAASFFSDFGLMWYLEELKKEEFRKFKEHLKQMTLQLELKQIPWTEVKKASREELANLLIKHYEEQQAWNITLRIFQKMDRKDLCMKVMRERTGYTKTYQAHAKQKFSRLWSSKSVTEIHLYFEEEVKQEECDHLDRLFAPKEAGKQPRTVIIQGPQGIGKTTLLMKLMMAWSDNKIFRDRFLYTFYFCCRELRELPPTSLADLISREWPDPAAPITEIVSQPERLLFVIDSFEELQGGLNEPDSDLCGDLMEKRPVQVLLSSLLRKKMLPEASLLIAIKPVCPKELRDQVTISEIYQPRGFNESDRLVYFCCFFKDPKRAMEAFNLVRESEQLFSICQIPLLCWILCTSLKQEMQKGKDLALTCQSTTSVYSSFVFNLFTPEGAEGPTPQTQHQLKALCSLAAEGMWTDTFEFCEDDLRRNGVVDADIPALLGTKILLKYGERESSYVFLHVCIQEFCAALFYLLKSHLDHPHPAVRCVQELLVANFEKARRAHWIFLGCFLTGLLNKKEQEKLDAFFGFQLSQEIKQQIHQCLKSLGERGNPQGQVDSLAIFYCLFEMQDPAFVKQAVNLLQEANFHIIDNVDLVVSAYCLKYCSSLRKLCFSVQNVFKKEDEHSSTSDYSLICWHHICSVLTTSGHLRELQVQDSTLSESTFVTWCNQLRHPSCRLQKLGINNVSFSGQSVLLFEVLFYQPDLKYLSFTLTKLSRDDIRSLCDALNYPAGNVKELALVNCHLSPIDCEVLAGLLTNNKKLTYLNVSCNQLDTGVPLLCEALCSPDTVLVYLMLAFCHLSEQCCEYISEMLLRNKSVRYLDLSANVLKDEGLKTLCEALKHPDCCLDSLCLVKCFITAAGCEDLASALISNQNLKILQIGCNEIGDVGVQLLCRALTHTDCRLEILGLEECGLTSTCCKDLASVLTCSKTLQQLNLTLNTLDHTGVVVLCEALRHPECALQVLGLRKTDFDEETQALLTAEEERNPNLTITDDCDTITRVEI

SEQ ID NO: 15 - DNA, 1899 bp, Homo sapiens
agcgcgcgactttttgaaagccaggagggttcgaattgcaacggcagctgccgggcgtatgtgttggtgctagaggcagctgcagggtctcgctgggggccgctcgggaccaattttgaagaggtacttggccacgacttattttcacctccgacctttccttccaggcggtgagactctggactgagagtggctttcacaatggaagggatcagtaatttcaagacaccaagcaaattatcagaaaaaaagaaatctgtattatgttcaactccaactataaatatcccggcctctccgtttatgcagaagcttggctttggtactggggtaaatgtgtacctaatgaaaagatctccaagaggtttgtctcattctccttgggctgtaaaaaagattaatcctatatgtaatgatcattatcgaagtgtgtatcaaaagagactaatggatgaagctaagattttgaaaagccttcatcatccaaacattgttggttatcgtgcttttactgaagccaatgatggcagtctgtgtcttgctatggaatatggaggtgaaaagtctctaaatgacttaatagaagaacgatataaagccagccaagatccttttccagcagccataattttaaaagttgctttgaatatggcaagagggttaaagtatctgcaccaagaaaagaaactgcttcatggagacataaagtcttcaaatgttgtaattaaaggcgattttgaaacaattaaaatctgtgatgtaggagtctctctaccactggatgaaaatatgactgtgactgaccctgaggcttgttacattggcacagagccatggaaacccaaagaagctgtggaggagaatggtgttattactgacaaggcagacatatttgcctttggccttactttgtgggaaatgatgactttatcgattccacacattaatctttcaaatgatgatgatgatgaagataaaacttttgatgaaagtgattttgatgatgaagcatactatgcagcgttgggaactaggccacctattaatatggaagaactggatgaatcataccagaaagtaattgaactcttctctgtatgcactaatgaagaccctaaagatcgtccttctgctgcacacattgttgaagctctggaaacagatgtctagtgatcatctcagctgaagtgtggcttgcgtaaataactgtttattccaaaatatttacatagttactatcagtagttattagactctaaaattggcatatttgaggaccatagtttcttgttaacatatggataactatttctaatatgaaatatgcttatattggctataagcacttggaattgtactgggttttctgtaaagttttagaaactagctacataagtactttgatactgctcatgctgacttaaaacactagcagtaaaacgctgtaaactgtaacattaaattgaatgaccattacttttattaatgatctttcttaaatattctatattttaatggatctactgacattagcactttgtacagtacaaaataaagtctacatttgtttaaaacactgaaccttttgctgatgtgtttatcaaatgataactggaagctgaggagaatatgcctcaaaaagagtagctccttggatacttcagactctggttacagattgtcttgatctcttggatctcctcagatctttggtttttgctttaatttattaaatgtattttccatactgagtttaaaatttattaatttgtaccttaagcatttcccagctgtgtaaaaacaataaaactcaaataggatgataaagaataaaggacactttgggtaccagaaaaaaaaaaaaaaaaaaaaaaaaaaaaaaaaaaaaaaaaaaaaaaa

SEQ ID NO: 16 - protein, 322 aa, Homo sapiens
MEGISNFKTPSKLSEKKKSVLCSTPTINIPASPFMQKLGFGTGVNVYLMKRSPRGLSHSPWAVKKINPICNDHYRSVYQKRLMDEAKILKSLHHPNIVGYRAFTEANDGSLCLAMEYGGEKSLNDLIEERYKASQDPFPAAIILKVALNMARGLKYLHQEKKLLHGDIKSSNVVIKGDFETIKICDVGVSLPLDENMTVTDPEACYIGTEPWKPKEAVEENGVITDKADIFAFGLTLWEMMTLSIPHINLSNDDDDEDKTFDESDFDDEAYYAALGTRPPINMEELDESYQKVIELFSVCTNEDPKDRPSAAHIVEALETDV

SEQ ID NO: 17 - DNA, 472 bp, Homo sapiens
gtcaccaggagggtatgcatagggagggcaagagctctgggccactgcgaagattcaaaagctccaaaaacctactgtagacatcgaagaaccaatatatacaatgggccaacaatccagtgtccgcaggctgaagaggagcgtcccctgtgaatccaacgaggccaacgaggccaatgaggccaacaagacgatgccggagaccccaactggggactcagacccgcaacctgctcctaaaaaaatgaaaacatctgagtcctcgaccatactagtggttcgctacaggaggaacgtgaaaagaacatctccagaggaactggtgaatgaccacgcccgagagaacagaatcaaccccgaccaaatggaggaggaggaattcatagaaataacgactgaaagacctaaaaagtagcaagaagctacatccctcaaacttcggcaatgaaaataaagtttgagaagctgaaaa

SEQ ID NO: 18 - protein, 103 aa, Homo sapiens
MGQQSSVRRLKRSVPCESNEANEANEANKTMPETPTGDSDPQPAPKKMKTSESSTILVVRYRRNVKRTSPEELVNDHARENRINPDQMEEEEFIEITTERPKK